(19) United States
(12) Patent Application Publication
(10) Pub. No.: US 2004/0096445 A1
(43) Pub. Date: May 20, 2004

(54) Subunit vaccines with A2 supermotifs

(76) Inventors: John Sidney, San Diego, CA (US); Alessandro Sette, La Jolla, CA (US); Howard M. Grey, La Jolla, CA (US); Scott Southwood, Santee, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/935,476

(22) Filed: Aug. 22, 2001

Related U.S. Application Data
(63) Continuation-in-part of application No. 09/346,105, filed on Jun. 30, 1999, now abandoned.
(60) Provisional application No. 60/264,969, filed on Jan. 29, 2001.

(51) Int. Cl. G01N 33/53; G01N 33/542; A61K 39/395

(52) U.S. Cl. 424/144.1; 435/7.9

(57) ABSTRACT

Methods to design vaccines which are effective in individuals bearing A2 supertype alleles are described. Single amino acid substitution analogs of known A2-supertype binding peptides, and large peptide libraries were utilized to rigorously define the peptide binding specificities of A2-supertype molecules. While each molecule was noted to have unique preferences, large overlaps in specificity were found. The presence of the hydrophobic and aliphatic residues L, I, V, M, A, T, and Q in position 2 of peptide ligands was commonly tolerated by A2-supertype molecules. L, I, V, M, A, and T were tolerated at the C-terminus. While examination of secondary influences on peptide binding revealed allele specific preferences, shared features could also be identified, and were utilized to define an A2-supermotif. Shared features also correlate with cross-reactivity; over 70% of the peptides that bound A*0201 with high affinity were found to bind at least 2 other A2-supertype molecules. Finally, the coefficients for use in the development of algorithms for the prediction of peptide binding to A2-supertype molecules are provided.

### CROSS-REFERENCE TO RELATED APPLICATIONS

This application is a continuation-in-part of U.S. application Ser. No. 09/346,105, entitled “Consistent Immune Responses in Diverse Genetic Populations,” filed Jun. 30, 1999, Sidney et al. This application also claims the benefit of the Jan. 29, 2001 filing date of U.S. Application Serial No. 60/264,969, entitled “Subunit Vaccines with A2 Supermotifs,” Sidney et al., each of which is incorporated by reference in its entirety.

### FEDERALLY SPONSORED RESEARCH AND DEVELOPMENT

Subject matter disclosed herein was funded, in part, by the United States government under grants from the National Institutes of Health. The U.S. government may have certain rights in this invention.

### TECHNICAL FIELD

Subject matter disclosed herein relates to the design of vaccines which will be effective in large portions of the population, in particular, those members of the population who are characterized as having an A2 supertype allele. Subunit vaccines which comprise the A2 supermotif can be designed to effect such population coverage.

### BACKGROUND

The genetic makeup of a given mammal encodes the structures associated with the immune system of that species. Although there is a great deal of genetic diversity in the human population, even more so comparing humans and other species, there are also common features and effects. In mammals, certain molecules associated with immune function are termed the major histocompatibility complex.

MHC molecules are classified as either Class I or Class II molecules. Class II MHC molecules are expressed primarily on cells involved in initiating and sustaining immune responses, such as T lymphocytes, B lymphocytes, macrophages, etc. Class II MHC molecules are recognized by helper T lymphocytes and induce proliferation of helper T lymphocytes and amplification of the immune response to the particular immunogenic peptide that is displayed. Class I MHC molecules are expressed on almost all nucleated cells and are recognized by cytotoxic T lymphocytes (CTLs), which then destroy the antigen-bearing cells. CTLs are particularly important in tumor rejection and in fighting viral infections.

CTLs recognize the antigen in the form of a peptide fragment bound to the MHC class I molecules rather than the intact foreign antigen itself. The antigen must normally be endogenously synthesized by the cell, and a portion of the protein antigen is degraded into small peptide fragments in the cytoplasm. Some of these small peptides translocate into a pre-Golgi compartment and interact with class I heavy chains to facilitate proper folding and association with the subunit β2 microglobulin. The peptide-MHC class I complex is then routed to the cell surface for expression and potential recognition by specific CTLs.

Investigations of the crystal structure of the human MHC class I molecule, HLA-A2.1, indicate that a peptide binding groove is created by the folding of the α1 and α2 domains of the class I heavy chain (Bjorkman, et al., Nature 329:506 (1987)). In these investigations, however, the identity of peptides bound to the groove was not determined.

Buus, et al., Science 242:1065 (1988) first described a method for acid elution of bound peptides from MHC. Subsequently, Rammensee and his coworkers (Falk, et al., Nature 351:290 (1991)) have developed an approach to characterize naturally processed peptides bound to class I molecules. Other investigators have successfully achieved direct amino acid sequencing of the more abundant peptides in various HPLC fractions by conventional automated sequencing of peptides eluted from class I molecules of the B type (Jardetzky, et al., Nature 353:326 (1991)) and of the A2.1 type by mass spectrometry (Hunt, et al., Science 225:1261 (1992)). A review of the characterization of naturally processed peptides in MHC Class I has been presented by Rötzschke & Falk (Rötzschke & Falk, Immunol. Today 12:447 (1991)). PCT publication WO 97/34621, incorporated herein by reference, describes peptides which have a binding motif for A2.1 alleles.

Sette, et al., Proc. Nat'l. Acad. Sci. USA 86:3296 (1989) showed that MHC allele specific motifs could be used to predict MHC binding capacity. Schaeffer, et al., Proc. Nat'l. Acad. Sci. USA 86:4649 (1989) showed that MHC binding was related to immunogenicity. Others (De Bruijn, et al., Eur. J Immunol., 21:2963-2970 (1991); Pamer, et al., 991 Nature 353:852-955 (1991)) have provided preliminary evidence that class I binding motifs can be applied to the identification of potential immunogenic peptides in animal models. Class I motifs specific for a number of human alleles of a given class I isotype have yet to be described. It is desirable that the combined frequencies of these different alleles should be high enough to cover a large fraction or perhaps the majority of the human outbreed population.

Despite the developments in the art, the prior art has yet to provide a useful human peptide-based vaccine or therapeutic agent based on this work.

### SUMMARY

The invention provides the parameters for the design of vaccines which are expected to effectively target large portions of the population. Following the guidance set forth herein, to prepare vaccines with respect to a particular infectious organism or virus or tumor, the relevant antigen is assessed to determine the location of epitopes which are most likely to effect a cytotoxic T response to an infection or tumor. By analyzing the amino acid sequence of the antigen according to the methods set forth herein, an appropriate set of epitopes can be identified. Peptides which consist of these epitopes can readily be tested for their ability to bind one or more than one HLA characteristic of the A2 supertype. In general, peptides which bind with an affinity represented by an IC₅₀ of 500 nM or less have a high probability of eliciting a cytotoxic T lymphocyte (CTL) response. The ability of these peptides to do so can also readily be verified. Vaccines can then be designed based on the immunogenic peptides thus identified. The vaccines themselves can consist of the peptides per se, precursors which will be expected to generate the peptides in vivo, or nucleic acids encoding these peptides for production in vivo.

Thus, in one aspect, the invention is directed to a method for identifying an epitope in an antigen characteristic of a pathogen or of a tumor which epitope is more likely to enhance an immune response in an individual bearing an allele of the A2 supertype than an arbitrarily chosen peptide, which method comprises analyzing the amino acid sequence of the antigen for segments of 8-11 amino acids where the segment at position 2 is a small or aliphatic hydrophobic residue (A, I, V, L, M or T) and the amino acid at the C-terminus of the segment is also a small or aliphatic hydrophobic residue selected from the same group. In preferred embodiments, the residue at position 2 is L or M. In other preferred embodiments, the segment contains 9-10 amino acids. In another preferred embodiment, the segment contains Q or N at position 1 and/or R, H or K at position 8, and lacks a D, E and G at position 3 when the segment is a 10-mer. Also preferred are V at position 2 and at the C-terminus.

Also described herein are compositions comprising immunogenic peptides having binding motif subsequences for HLA-A2.1 molecules. The immunogenic epitopes in the peptides, which bind to the appropriate MHC allele, are preferably 8-11 residues in length and more preferably 9 to 10 residues in length and comprise conserved residues at certain positions such as positions 2 and the C-terminus. Moreover, the peptides do not comprise negative binding residues as defined herein at other positions such as positions 1, 3, 6 and/or 7 in the case of peptides 9 amino acids in length and positions 1, 3, 4, 5, 7, 8 and/or 9 in the case of peptides 10 amino acids in length. The present invention defines positions within a motif enabling the selection of peptides which will bind efficiently to HLA A2.1.

Epitopes on a number of immunogenic target proteins can be identified using the sequence motifs described herein. Examples of suitable antigens include prostate cancer specific antigen (PSA), hepatitis B core and surface antigens (HBVc, HBVs) hepatitis C antigens, Epstein-Barr virus antigens, human immunodeficiency type-1 virus (HIV1), Kaposi's sarcoma herpes virus (KSHV), human papilloma virus (HPV) antigens, Lassa virus, mycobacterium tuberculosis (MT), p53, CEA, trypanosome surface antigen (TSA) and Her2/neu. The peptides and nucleic acids encoding them are useful in pharmaceutical compositions for both in vivo and ex vivo therapeutic and diagnostic applications.

Definitions

The term “peptide” is used interchangeably with “oligopeptide” in the present specification to designate a series of residues, typically L-amino acids, connected one to the other typically by peptide bonds between the alpha-amino and carbonyl groups of adjacent amino acids. The oligopeptides are generally less than 250 amino acids in length, and can be less than 150, 100, 75, 50, 25, or 15 amino acids in length. Further, an oligopeptide of the invention can be such that it does not comprise more than 15 contiguous amino acids of a native antigen.

The nomenclature used to describe peptide compounds follows the conventional practice where the amino group is presented to the left (the N-terminus) and the carboxyl group to the right (the C-terminus) of each amino acid residue. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G.

An “immunogenic peptide” or “epitope” is a peptide or amino acid sequence which comprises an allele-specific motif such that the peptide sequence will bind an MHC molecule and induce a CTL response. Immunogenic peptides of the invention are capable of binding to an appropriate HLA-A2 molecule and inducing a cytotoxic T-cell response against the antigen from which the immunogenic peptide is derived. The immunogenic peptides of the invention are less than about 15 residues in length, often less than 12 residues in length and usually consist of between about 8 and about 11 residues, preferably 9 or 10 residues.

Immunogenic peptides are conveniently identified using the algorithms of the invention. The algorithms are mathematical procedures that produce a score which enables the selection of immunogenic peptides. Typically one uses the algorithmic score with a “binding threshold” to enable selection of peptides that have a high probability of binding at a certain affinity and will in turn be immunogenic. The algorithm is based upon either the effects on MHC binding of a particular amino acid at a particular position of a peptide or the effects on binding of a particular substitution in a motif containing peptide.

Binding results are often expressed in terms of “IC₅₀'s.” IC₅₀ is the concentration of peptide in a binding assay at which 50% inhibition of binding of a reference peptide is observed. Given the conditions in which the assays as described herein are run (i.e., limiting HLA proteins and labeled peptide concentrations), these values approximate K_{D} values. Assays for determining binding are described in detail in PCT publications WO 94/20127 and WO 94/03205. It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand and therefore not reflect the true K_{D} value.

Binding is often expressed as a ratio relative to a reference peptide. As a particular assay becomes more, or less, sensitive, the IC₅₀'s of the peptides tested may change somewhat. However, the binding relative to the reference peptide will not significantly change. For example, in an assay run under conditions such that the IC₅₀ of the reference peptide increases 10-fold, the IC₅₀ values of the test peptides will also shift approximately 10-fold. Therefore, to avoid ambiguities, the assessment of whether a peptide is a good, intermediate, weak, or negative binder is generally based on its IC₅₀, relative to the IC₅₀ of a standard peptide. The binding may be reported as a ratio or the ratio may be used to normalize the IC₅₀ value as described in Example 1.

As used herein, high affinity with respect to HLA class I molecules is defined as binding with an IC₅₀ or K_{D} value of less than 50 nM. Intermediate affinity is binding with an IC₅₀ (or K_{D}) of between about 50 and about 500 nM.

A “conserved residue” is an amino acid which occurs in a significantly higher frequency than would be expected by random distribution at a particular position in a peptide. Typically a conserved residue is one where the MHC structure may provide a contact point with the immunogenic peptide. One to three, preferably two, conserved residues within a peptide of defined length defines a motif for an immunogenic peptide. These residues are typically in close contact with the peptide binding groove, with their side chains buried in specific pockets of the groove itself. Typically, an immunogenic peptide will comprise up to three conserved residues, more usually two conserved residues.

As used herein, “negative binding residues” are amino acids which if present at certain positions (for example, positions 1, 3 and/or 7 of a 9-mer) will result in a peptide being a nonbinder or poor binder and in turn fail to be immunogenic i.e. induce a CTL response.

The term “motif” refers to the pattern of residues in a peptide of defined length, usually about 8 to about 11 amino acids, which is recognized by a particular MHC allele. The peptide motifs are typically different for each human MHC allele and differ in the pattern of the highly conserved residues and negative residues.

The binding motif for an allele can be defined with increasing degrees of precision. In one case, all of the conserved residues are present in the correct positions in a peptide and there are no negative residues in positions 1, 3 and/or 7.

A “supermotif” is a peptide binding specificity shared by HLA molecules encoded by two or more HLA alleles. A supermotif-bearing epitope preferably is recognized with high or intermediate affinity (as defined herein) by two or more HLA antigens.

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, and HLA xx-like supertype molecules (where xx denotes a particular HLA type) are synonyms.

The phrases “isolated” or “biologically pure” refer to material which is substantially or essentially free from components which normally accompany it as found in its native state. Thus, the peptides of this invention do not contain materials normally associated with their in situ environment, e.g., MHC I molecules on antigen presenting cells. Even where a protein has been isolated to a homogenous or dominant band, there are trace contaminants in the range of 5-10% of native protein which co-purify with the desired protein. Isolated peptides of this invention do not contain such endogenous co-purified protein.

The term “residue” refers to an amino acid or amino acid mimetic incorporated in an oligopeptide by an amide bond or amide bond mimetic.

### BRIEF DESCRIPTION OF THE DRAWINGS

FIG. 1. Position 2 and C-terminus fine specificity of HLA-A*0201. The preference for specific residues in position 2 (a) or at the C-terminus (b) is shown at a function of the percent of peptides bearing a specific residue that bind A*0201 with IC50 of 500 nM or better. ARB values of peptides bearing specific residues in position 2 (a) or at the C-terminus (b) were calculated as described herein, and indexed relative to the residue with the highest binding capacity. The average (geometric) binding capacity of peptides with L in position 2 was 1991 nM. The average (geometric) binding capacity of peptides with V at the C-terminus was 2133 nM. Peptides included in the analysis had at least one tolerated anchor residue, as described in the text, at either position 2 or the C-terminus.

FIG. 2. Map of the A*0201 motif. Summary map of the A*0201 motif for 8-mer (b), 10-mer (c) and 11-mer (d) peptides. At secondary anchor positions, residues shown as preferred (or deleterious) are associated with an average binding capacity at least 3-fold greater than (or 3-fold less than) peptides of the same size carrying other residues at the same position. At the primary anchor positions, preferred residues are those associated with an average binding capacity within 10-fold of the optimal residue at the same position. Tolerated primary anchor residues are those associated with an average binding capacity between 10- and 100-fold of the optimal residue at the same position.

FIG. 3. Position 2 fine specificity of HLA-A2-supertype molecules. ARB values of peptides bearing specific residues in position 2 were calculated for each A2-supertype molecule as described in the text, and indexed relative to the residue with the highest ARB for each specific molecule. The average (geometric) binding capacity of the peptides bearing the residue with the highest ARB were 55, 59, 89, and 41 nM for A*0202, A*0206, and A*6802, respectively.

FIG. 4. C-terminal fine specificity of HLA-A2-supertype molecules. ARB values of peptides bearing specific residues at the C-terminus were calculated for each A2-supertype molecule as described in the text, and indexed relative to the residue with the highest ARB for each specific molecule. The average (geometric) binding capacity of the peptides bearing the residue with the highest ARB were 291, 48, 250, and 553 nM for A*0202, A*0203, A*0206, and A*6802, respectively.

FIG. 5. Map of the A*0202 motif. Summary map of A*0202 motif for 9-mer (a) and 10-mer (b) peptides. At secondary anchor positions, residues shown as preferred (or deleterious) are associated with an average binding capacity at least 3-fold greater than (or 3-fold less than) peptides of the same size carrying other residues at the same position. At the primary anchor positions, preferred residues are those associated with an average binding capacity within 10-fold of the optimal residue at the same position. Tolerated primary anchor residues are those associated with an average binding capacity between 10- and 100-fold of the optimal residue at the same position.

FIG. 6. Map of the A*0203 motif. Summary maps of A*0203 motif for 9-mer (a) and 10-mer (b) peptides. At secondary anchor positions, residues shown as preferred (or deleterious) are associated with an average binding capacity at least 3-fold greater than (or 3-fold less than) peptides of the same size carrying other residues at the same position. At the primary anchor positions, preferred residues are those associated with an average binding capacity within 10-fold of the optimal residue at the same position. Tolerated primary anchor residues are those associated with an average binding capacity between 10- and 100-fold of the optimal residue at the same position.

FIG. 7. Map of the A*0206 motif. Summary maps of A*0206 motif for 9-mer (a) and 10-mer (b) peptides. At secondary anchor positions, residues shown as preferred (or deleterious) are associated with an average binding capacity at least 3-fold greater than (or 3-fold less than) peptides of the same size carrying other residues at the same position. At the primary anchor positions, preferred residues are those associated with an average binding capacity within 10-fold of the optimal residue at the same position. Tolerated primary anchor residues are those associated with an average binding capacity between 10- and 100-fold of the optimal residue at the same position.

FIG. 8. Map of the A*6802 motif. Summary maps of A*6802 motif for 9-mer (a) and 10-mer (b) peptides. At secondary anchor positions, residues shown as preferred (or deleterious) are associated with an average binding capacity at least 3-fold greater than (or 3-fold less than) peptides of the same size carrying other residues at the same position. At the primary anchor positions, preferred residues are those associated with an average binding capacity within 10-fold of the optimal residue at the same position. Tolerated primary anchor residues are those associated with an average binding capacity between 10- and 100-fold of the optimal residue at the same position.

FIG. 9. A2 supermotif consensus summary of secondary and primary anchor influences on A2-supertype binding capacity of 9-(a) and 10-mer (b) peptides. Residues shown significantly influence binding to 3 or more A2-supertype molecules. The number of molecules influenced are indicated in parentheses. At secondary anchor positions, residues are considered preferred only if they do not have a deleterious influence on more than one molecule. Preferred residues which were deleterious in the context of one molecule are indicated by reduced and italicized font. Assessment at the primary anchor positions are based on single substitution and peptide library analyses, as discussed in the text.

### DESCRIPTION OF THE PREFERRED EMBODIMENTS

The present invention relates in part to an epitope-based approach for vaccine design. Such an approach is based on the well-established finding that the mechanism for inducing CTL immune response comprises the step of presenting a CTL epitope as a peptide of about 8-11 amino acids bound to an HLA molecule displayed on an antigen-presenting cell. The HLA molecule is the product of a class I MHC wherein the product is expressed on most nucleated cells.

The products of the MHC class I alleles are generically characterized as A, B and C HLA molecules. Within each of these categories, there is a multiplicity of allelic variants in the population; indeed, there are believed to be well over 500 class I and class II alleles. Since a cytotoxic T-cell response cannot be elicited unless the epitope is presented by the class I HLA contained on the surface of the cells of the individual to be immunized, it is important that the epitope be one that is capable of binding the HLA exhibited by that individual.

The starting point, therefore, for the design of effective vaccines is to ensure that the vaccine will generate a large number of epitopes that can successfully be presented. It may be possible to administer the peptides representing the epitopes per se. Such administration is dependent on the presentation of “empty” HLA molecules displayed on the cells of the subject. In one approach to use of the immunogenic peptides per se, these peptides may be incubated with antigen-presenting cells from the subject to be treated ex vivo and the cells then returned to the subject.

Alternatively, the 8-11 amino acid peptide can be generated in situ by administering a nucleic acid containing a nucleotide sequence encoding it. Means for providing such nucleic acid molecules are described in WO 99/58658, the disclosure of which is incorporated herein by reference. Further, the immunogenic peptides can be administered as portions of a larger peptide molecule and cleaved to release the desired peptide. The larger peptide may contain extraneous amino acids, in general the fewer the better. Thus, peptides which contain such amino acids are typically 25 amino acids or less, more typically 20 amino acids or less, and more typically 15 amino acids or less. The precursor may also be a heteropolymer or homopolymer containing a multiplicity of different or same CTL epitopes. Of course, mixtures of peptides and nucleic acids which generate a variety of immunogenic peptides can also be employed. The design of the peptide vaccines, the nucleic acid molecules, or the hetero- or homo-polymers is dependent on the inclusion of the desired epitope. The present invention provides a paradigm for identifying the relevant epitope which is effective across the broad population range of individuals who are characterized by the A2 supertype. The following pages describe the methods and results of experiments for identification of the A2 supermotif.

It is preferred that peptides include an epitope that binds to an HLA-A2 supertype allele. These motifs may be used to define T-cell epitopes from any desired antigen, particularly those associated with human viral diseases, cancers or autoimmune diseases, for which the amino acid sequence of the potential antigen or autoantigen targets is known.

Epitopes on a number of potential target proteins can be identified based upon HLA binding motifs. Examples of suitable antigens include prostate specific antigen (PSA), hepatitis B core and surface antigens (HBVc, HBVs) hepatitis C antigens, Epstein-Barr virus antigens, melanoma antigens (e.g., MAGE-1), human immunodeficiency virus (HIV) antigens, human papilloma virus (HPV) antigens, p53, CEA, trypanosome surface antigen (TSA), and Her2/neu.

Peptides comprising the epitopes from these antigens may be synthesized and then tested for their ability to bind to the appropriate MHC molecules in assays using, for example, purified class I molecules and radioiodonated peptides and/or cells expressing empty class I molecules by, for instance, immunofluorescent staining and flow microfluorometry, peptide-dependent class I assembly assays, and inhibition of CTL recognition by peptide competition. Those peptides that bind to the class I molecule may be further evaluated for their ability to serve as targets for CTLs derived from infected or immunized individuals, as well as for their capacity to induce primary in vitro or in vivo CTL responses that can give rise to CTL populations capable of reacting with virally infected target cells or tumor cells as potential therapeutic agents.

The MHC class I antigens are encoded by the HLA-A, B, and C loci. HLA-A and B antigens are expressed at the cell surface at approximately equal densities, whereas the expression of HLA-C is significantly lower (perhaps as much as 10-fold lower). Each of these loci have a number of alleles. The peptide binding motifs of the invention are relatively specific for each allelic subtype.

For peptide-based vaccines, peptides preferably comprise a motif recognized by an MHC I molecule having a wide distribution in the human population, or comprise a motif recognized by a genetically diverse population. Since the MHC alleles occur at different frequencies within different ethnic groups and races, the choice of target MHC allele may depend upon the target population. Table 1 shows the frequency of various alleles at the HLA-A locus products among different races. For instance, the majority of the Caucasoid population can be covered by peptides which bind to four HLA-A allele subtypes, specifically HLA-A2.1, A1, A3.2, and A24.1. Similarly, the majority of the Asian population is encompassed with the addition of peptides binding to a fifth allele HLA-A11.2.

TABLE 1

| A Allele/Subtype | N(69)* | A(54) | C(502) |
|---|---|---|---|
| A1 | 10.1(7) | 1.8(1) | 27.4(138) |
| A2.1 | 11.5(8) | 37.0(20) | 39.8(199) |
| A2.2 | 10.1(7) | 0 | 3.3(17) |
| A2.3 | 1.4(1) | 5.5(3) | 0.8(4) |
| A2.4 | — | — | — |
| A2.5 | — | — | — |
| A3.1 | 1.4(1) | 0 | 0.2(0) |
| A3.2 | 5.7(4) | 5.5(3) | 21.5(108) |
| A11.1 | 0 | 5.5(3) | 0 |
| A11.2 | 5.7(4) | 31.4(17) | 8.7(44) |
| A11.3 | 0 | 3.7(2) | 0 |
| A23 | 4.3(3) | — | 3.9(20) |
| A24 | 2.9(2) | 27.7(15) | 15.3(77) |
| A24.2 | — | — | — |
| A24.3 | — | — | — |
| A25 | 1.4(1) | — | 6.9(35) |
| A26.1 | 4.3(3) | 9.2(5) | 5.9(30) |
| A26.2 | 7.2(5) | — | 1.0(5) |
| A26V | — | 3.7(2) | — |
| A28.1 | 10.1(7) | — | 1.6(8) |
| A28.2 | 1.4(1) | — | 7.5(38) |
| A29.1 | 1.4(1) | — | 1.4(7) |
| A29.2 | 10.1(7) | 1.8(1) | 5.3(27) |
| A30.1 | 8.6(6) | — | 4.9(25) |
| A30.2 | 1.4(1) | — | 0.2(1) |
| A30.3 | 7.2(5) | — | 3.9(20) |
| A31 | 4.3(3) | 7.4(4) | 6.9(35) |
| A32 | 2.8(2) | — | 7.1(36) |
| Aw33.1 | 8.6(6) | — | 2.5(13) |
| Aw33.2 | 2.8(2) | 16.6(9) | 1.2(6) |
| Aw34.1 | 1.4(1) | — | — |
| Aw34.2 | 14.5(10) | — | 0.8(4) |
| Aw36 | 5.9(4) | — | — |

Table compiled from B. DuPont, Immunobiology of HLA, Vol. I, Histocompatibility Testing 1987, Springer-Verlag, New York 1989.
*N = Negroid; A = Asian; C = Caucasoid. Numbers in parenthesis represent the number of individuals included in the analysis.

Cross-reactive binding of HLA-A2.1 motif-bearing peptides with other HLA-A2 allele-specific molecules can occur. Those allele-specific molecules that share binding specificities with HLA-A2.1 are deemed to comprise the HLA-A2.1 supertype. The B pocket of A2 supertype HLA molecules is characterized by a consensus motif including residues (this nomenclature uses single letter amino acid codes, where the subscript indicates peptide position) F/Y₉, A₂₄, M₄₅, E/N₆₃, K/N₆₆, V₆₇, H/Q₇₀ and Y/C₉₉. Similarly, the A2-supertype F pocket is characterized by a consensus motif including residues D₇₇, T₈₀, L₈₁ and Y₁₁₆ (155). About 66% of the peptides binding A*0201 will be cross-reactive amongst three or more A2-supertype alleles.

The A2 supertype as defined herein is consistent with cross-reactivity data, (Fruci, D. et al., Hum. Immunol. 38:187, 1993), from live cell binding assays (del Guercio, M.-F. et al., J. Immunol. 154:685, 1995) and data obtained by sequencing naturally processed peptides (Sudo, T., et al., J. Immunol. 155:4749, 1995) bound to HLA-A2 allele-specific molecules. Accordingly the family of HLA molecules (i.e., the HLA-A2 supertype that binds these peptides) is comprised of at least nine HLA-A proteins: A*0201, A*0202, A*0203, A*0204, A*0205, A*0206, A*0207, A*6802, and A*6901.

As described herein, the HLA-A2 supermotif comprises peptide ligands with L, I, V, M, A, T, or Q as primary anchor residues at position 2 and L, I, V, M, A, or T as a primary anchor residue at the C-terminal position of the epitope. HLA-A2 motifs that are most particularly relevant to the invention claimed here comprise V, A, T, or Q at position two and L, I, V, M, A, or T at the C-terminal anchor position. A peptide epitope comprising an HLA-A2 supermotif may bind more than one HLA-A2 supertype molecule.

A procedure which may be used to identify peptides of the present invention is disclosed in Falk, et al., Nature 351:290 (1991), incorporated herein by reference. Briefly, the methods involve large-scale isolation of MHC class I molecules, typically by immunoprecipitation or affinity chromatography, from appropriate cell or cell line. Examples of other methods for isolation of the desired MHC molecule equally well known to the artisan include ion exchange chromatography, lectin chromatography, size exclusion, high performance ligand chromatography, and a combination of all of the above techniques.

In a typical case, immunoprecipitation may be used to isolate the desired allele. A number of protocols can be used, depending upon the specificity of the antibodies used. For example, allele-specific mAb reagents can be used for the affinity purification of the HLA-A, HLA-B1, and HLA-C molecules. Several mAb reagents for the isolation of HLA-A molecules are available. The monoclonal BB7.2 is suitable for isolating HLA-A2 molecules. Affinity columns prepared with these mAbs using standard techniques are successfully used to purify the respective HLA-A allele products.

In addition to allele-specific mAbs, broadly reactive anti-HLA-A, B, C mAbs, such as W6/32 and B9.12.1, and one anti-HLA-B, C mAb, B1.23.2, could be used in alternative affinity purification protocols as described in the example section below.

The peptides bound to the peptide binding groove of the isolated MHC molecules are eluted typically using acid treatment. Peptides can also be dissociated from class I molecules by a variety of standard denaturing means, such as heat, pH, detergents, salts, chaotropic agents, or a combination thereof.

Peptide fractions are further separated from the MHC molecules by reversed-phase high performance liquid chromatography (HPLC) and sequenced. Peptides can be separated by a variety of other standard means well known to the artisan, including filtration, ultrafiltration, electrophoresis, size chromatography, precipitation with specific antibodies, ion exchange chromatography, isoelectrofocusing, and the like.

Sequencing of the isolated peptides can be performed according to standard techniques such as Edman degradation (Hunkapiller, M. W., et al., Methods Enzymol. 91, 399 [1983]). Other methods suitable for sequencing include mass spectrometry sequencing of individual peptides as previously described (Hunt, et al., Science 225:1261 (1992), which is incorporated herein by reference). Amino acid sequencing of bulk heterogeneous peptides (e.g., pooled HPLC fractions) from different class I molecules typically reveals a characteristic sequence motif for each class I allele.

Definition of motifs specific for different class I alleles allows the identification of potential peptide epitopes from an antigenic protein whose amino acid sequence is known. Typically, identification of potential peptide epitopes is initially carried out using a computer to scan the amino acid sequence of a desired antigen for the presence of motifs.

Following identification of motif-bearing epitopes, the epitopic sequences are then synthesized. The capacity to bind MHC Class molecules is measured in a variety of different ways. One means is a Class I molecule binding assay as described in the related applications, noted below. Other alternatives described in the literature include inhibition of antigen presentation (Sette, et al., J. Immunol. 141:3893 (1991), in vitro assembly assays (Townsend, et al., Cell 62:285 (1990), and FACS based assays using mutated cells, such as RMA.S (Melief, et al., Eur. J. Immunol. 21:2963 (1991)).

As disclosed herein, higher HLA binding affinity is correlated with greater immunogenicity. Greater immunogenicity can be manifested in several different ways. Immunogenicity can correspond to whether an immune response is elicited at all, and to the vigor of any particular response, as well as to the extent of a diverse population in which a response is elicited. For example, a peptide might elicit an immune response in a diverse array of the population, yet in no instance produce a vigorous response. In accordance with the principles disclosed herein, close to 90% of high binding peptides have been found to be immunogenic, as contrasted with about 50% of the peptides which bind with intermediate affinity. Moreover, higher binding affinity peptides lead to more vigorous immunogenic responses. As a result, less peptide is required to elicit a similar biological effect if a high affinity binding peptide is used. Thus, in preferred embodiments of the invention, high affinity binding epitopes are particularly useful. Nevertheless, substantial improvements over the prior art are achieved with intermediate or high binding peptides.

The relationship between binding affinity for HLA class I molecules and immunogenicity of discrete peptide epitopes has been determined for the first time in the art by the present inventors. In these experiments, in which discrete peptides were referred to, it is to be noted that cellular processing of peptides in vivo will lead to such peptides even if longer fragments are used. Accordingly, longer peptides comprising one or more epitopes are within the scope of the invention. The correlation between binding affinity and immunogenicity was analyzed in two different experimental approaches (Sette, et al., J. Immunol. 153:5586-5592, 1994). In the first approach, the immunogenicity of potential epitopes ranging in HLA binding affinity over a 10,000-fold range was analyzed in HLA-A 0201 transgenic mice. In the second approach, the antigenicity of approximately 100 different hepatitis B virus (HBV)-derived potential epitopes, all carrying A*0201 binding motifs, was assessed by using PBL (peripheral blood lymphocytes) from acute hepatitis patients. Pursuant to these approaches, it was determined that an affinity threshold value of approximately 500 nM (preferably 50 nM or less) is correlated with the capacity of a peptide epitope to elicit a CTL response. These data are true for class I binding affinity measurements for naturally processed peptides and for synthesized T-cell epitopes. These data also indicate the important role of determinant selection in the shaping of T-cell responses (see, e.g., Schaeffer, et al., Proc. Natl. Acad. Sci. USA 86:4649-4653, 1989).

Accordingly, CTL-inducing peptides preferably include those that have an IC₅₀ for class I HLA molecules of 500 nM or less. In the case of motif-bearing peptide epitopes from tumor associated antigens, a binding affinity threshold of 200 nM has been shown to be associated with killing of tumor cells by resulting CTL populations.

In a preferred embodiment, following assessment of binding activity for an HLA-A2 allele-specific molecule, peptides exhibiting high or intermediate affinity are then considered for further analysis. Selected peptides may be tested on other members of the supertype family. In preferred embodiments, peptides that exhibit cross-reactive binding are then used in vaccines or in cellular screening analyses.

For example, peptides that test positive in the HLA-A2 binding assay, i.e., that have binding affinity values of 500 nM or less, are assayed for the ability of the peptides to induce specific CTL responses in vitro. For instance, antigen-presenting cells that have been incubated with a peptide can be assayed for the ability to induce CTL responses in responder cell populations. Antigen-presenting cells can be normal cells such as peripheral blood mononuclear cells or dendritic cells (Inaba, et al., J. Exp. Med. 166:182 (1987); Boog, Eur. J. Immunol. 18:219 [1988]).

Alternatively, mutant mammalian cell lines that are deficient in their ability to load class I molecules with internally processed peptides, such as the mouse cell lines RMA-S (Kärre, et al., Nature, 319:675 (1986); Ljunggren, et al., Eur. J. Immunol. 21:2963-2970 (1991)), and the human somatic T-cell hybrid, T-2 (Cerundolo, et al., Nature 345:449-452 (1990)) and which have been transfected with the appropriate genes which encode human class I molecules are conveniently used, when peptide is exogenously added to them, to test for the capacity of the peptide to induce in vitro primary CTL responses. Other eukaryotic cell lines that can be used include various insect cell lines such as mosquito larvae (ATCC cell lines CCL 125, 126, 1660, 1591, 6585, 6586), silkworm (ATTC CRL 8851), armyworm (ATCC CRL 1711), moth (ATCC CCL 80) and Drosophila cell lines such as a Schneider cell line (see Schneider J. Embryol. Exp. Morphol. 27:353-365 [1927]).

Peripheral blood lymphocytes are conveniently isolated following simple venipuncture or leukapheresis of normal donors or patients and used as the responder cell sources of CTL precursors. In one embodiment, the appropriate antigen-presenting cells are incubated with 10-100 μM of peptide in serum-free media for 4 hours under appropriate culture conditions. The peptide-loaded antigen-presenting cells are then incubated with the responder cell populations in vitro for 7 to 10 days under optimized culture conditions. Positive CTL activation can be determined by assaying the cultures for the presence of CTLs that kill radiolabeled target cells, both specific peptide-pulsed targets as well as target cells expressing endogenously processed form of the relevant virus or tumor antigen from which the peptide sequence was derived.

Specificity and MHC restriction of the CTL is determined by testing against different peptide target cells expressing appropriate or inappropriate human MHC class I. The peptides that test positive in the MHC binding assays and give rise to specific CTL responses are referred to herein as immunogenic peptides.

Kast, et al. (J. Immunol. 152:3904-3912, 1994) have shown that motif-bearing peptides account for 90% of the epitopes that bind to allele-specific HLA class I molecules. In this study all possible peptides of 9 amino acids in length and overlapping by eight amino acids (240 peptides), which cover the entire sequence of the E6 and E7 proteins of human papillomavirus type 16, were evaluated for binding to five allele-specific HLA molecules that are expressed at high frequency among different ethnic groups. This unbiased set of peptides allowed an evaluation of the predictive value of HLA class I motifs. From the set of 240 peptides, 22 peptides were identified that bound to an allele-specific HLA molecules with high or intermediate affinity. Of these 22 peptides, 20, (i.e. 91%), were motif-bearing. Thus, this study demonstrated the value of motifs for the identification of peptide epitopes for inclusion in a vaccine: application of motif-based identification techniques eliminates screening of 90% of the potential epitopes. The quantity of available peptides, and the complexity of the screening process would make a comprehensive evaluation of an antigen highly difficult, if not impossible without use of motifs.

An immunogenic peptide epitope of the invention may be included in a polyepitopic vaccine composition comprising additional peptide epitopes of the same antigen, antigens from the same source, and/or antigens from a different source. Moreover, class II epitopes can be included along with class I epitopes. Peptide epitopes from the same antigen may be adjacent epitopes that are contiguous in sequence or may be obtained from different regions of the protein.

As noted in greater detail below, the immunogenic peptides can be prepared synthetically, such as by chemical synthesis or by recombinant DNA technology, or isolated from natural sources such as whole viruses or tumors. Although the peptide will preferably be substantially free of other naturally occurring host cell proteins and fragments thereof, in some embodiments the peptides can be synthetically conjugated to native fragments or particles.

The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

Desirably, the peptide will be as small as possible while still maintaining substantially all of the biological activity of the large peptide. When possible, it may be desirable to optimize peptide epitopes of the invention to a length of 9 or 10 amino acid residues, commensurate in size with endogenously processed viral peptides or tumor cell peptides that are bound to MHC class I molecules on the cell surface.

Peptides having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T-cell. For instance, the peptides may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany and Merrifield, The Peptides, Gross and Meienhofer, eds. (N.Y., Academic Press), pp. 1-284 (1979); and Stewart and Young, Solid Phase Peptide Synthesis, (Rockford, Ill., Pierce), 2d Ed. (1984).

The peptides can also be modified by extending or decreasing the compound's amino acid sequence, e.g., by the addition or deletion of amino acids. The peptides or analogs of the invention can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, but may include non-natural amino acids as well, such as β-γ-δ-amino acids, as well as many derivatives of L-α-amino acids such as D-isomers of natural amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T-cell receptors. In addition, multiple substitutions using small, relatively neutral moieties such as Ala, Gly, Pro, or similar residues may be employed. The substitutions may produce multi-epitopic peptides which are homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T-cell receptor may also be achieved by such substitutions, compared to the affinity of the parent peptide. In any event, such substitutions generally employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions generally are made in accordance with the following Table 2 when it is desired to finely modulate the characteristics of the peptide.

TABLE 2

| Original Residue | Exemplary Substitution |
|---|---|
| Ala | Ser |
| Arg | Lys, His |
| Asn | Gln |
| Asp | Glu |
| Cys | Ser |
| Gln | Asn |
| Glu | Asp |
| Gly | Pro |
| His | Lys; Arg |
| Ile | Leu; Val |
| Leu | Ile; Val |
| Lys | Arg; His |
| Met | Leu; Ile |
| Phe | Tyr; Trp |
| Ser | Thr |
| Thr | Ser |
| Trp | Tyr; Phe |
| Tyr | Trp; Phe |
| Val | Ile; Leu |

Substantial changes in function (e.g., affinity for MHC molecules or T-cell receptors) are made by selecting substitutions that are less conservative than those in Table 2, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) hydrophilic residue, e.g. seryl, is substituted for (or by) a hydrophobic residue, e.g. leucyl, isoleucyl, phenylalanyl, valyl or alanyl; (b) a residue having an electropositive side chain, e.g., lysl, arginyl, or histidyl, is substituted for (or by) an electronegative residue, e.g. glutamyl or aspartyl; or (c) a residue having a bulky side chain, e.g. phenylalanine, is substituted for (or by) one not having a side chain, e.g., glycine.

The peptides may also comprise isosteres of two or more residues in the immunogenic peptide. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

Modifications of peptides with various amino acid mimetics or unnatural amino acids are particularly useful in increasing the stability of the peptide in vivo. Stability can be assayed in a number of ways. For instance, peptidases and various biological media, such as human plasma and serum, have been used to test stability. See, e.g., Verhoef, et al., Eur. J. Drug Metab. Pharmacokin. 11:291-302 (1986). Half life of the peptides of the present invention is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides of the present invention or analogs thereof which have CTL stimulating activity may be modified to provide desired attributes other than improved serum half life. For instance, the ability of the peptides to induce CTL activity can be enhanced by linkage to a sequence which contains at least one epitope that is capable of inducing a T helper cell response.

In some embodiments, the T helper peptide is one that is recognized by T helper cells in the majority of the population. This can be accomplished by selecting amino acid sequences that bind to many, most, or all of the MHC class II molecules. These are known as “loosely MHC-restricted” T helper sequences. Examples of amino acid sequences that are loosely MHC-restricted include sequences from antigens such as Tetanus toxin at positions 830-843 (QYIKANSKFIGITE), Plasmodium falciparum circumsporozoite (CS) protein at positions 378-398 (DIEKKIAKMEKASSVFNVVNS), and Streptococcus 18 kD protein at positions 1-16 (YGAVDSILGGVATYGAA).

Alternatively, it is possible to prepare synthetic peptides capable of stimulating T helper lymphocytes, in a loosely MHC-restricted fashion, using amino acid sequences not found in nature. These synthetic compounds, called Pan-DR-binding epitopes or PADRE™ molecules (Epimmune, San Diego, Calif.), are designed on the basis of their binding activity to most HLA-DR (human MHC class II) molecules (see, e.g., U.S. Pat. No. 5,736,142).

Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the CTL peptide may be linked to the T helper peptide without a spacer.

The immunogenic peptide may be linked to the T helper peptide, either directly or via a spacer, at the amino or carboxy terminus of the CTL peptide. The amino terminus of either the immunogenic peptide or the T helper peptide may be acylated.

Exemplary T helper peptides include tetanus toxoid 830-843, influenza 307-319, malaria circumsporozoite 382-398 and 378-389.

In some embodiments it may be desirable to include in the pharmaceutical compositions of the invention at least one component which primes CTL. Lipids have been identified as agents capable of priming CTL in vivo against viral antigens. For example, palmitic acid residues can be attached to the alpha and epsilon amino groups of a Lys residue and then linked, e.g., via one or more linking residues such as Gly, Gly-Gly-, Ser, Ser-Ser, or the like, to an immunogenic peptide. The lipidated peptide can then be injected directly in a micellar form, incorporated into a liposome or emulsified in an adjuvant, e.g., incomplete Freund's adjuvant. In a preferred embodiment a particularly effective immunogen comprises palmitic acid attached to alpha and epsilon amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide. See, Deres, et al., Nature 342:561-564 (1989). Peptides of the invention can be coupled to P₃CSS, for example, and the lipopeptide administered to an individual to specifically prime a CTL response to the target antigen. Further, as the induction of neutralizing antibodies can also be primed with P₃CSS conjugated to a peptide which displays an appropriate epitope, the two compositions can be combined to more effectively elicit both humoral and cell-mediated responses to infection.

In addition, additional amino acids can be added to the termini of a peptide to provide for ease of linking peptides one to another, for coupling to a carrier support, or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide. Modification at the C terminus in some cases may alter binding characteristics of the peptide. In addition, the peptide or oligopeptide sequences can differ from the natural sequence by being modified by terminal-NH2 acylation, e.g., by alkanoyl (C1-C20) or thioglycolyl acetylation, terminal-carboxyl amidation, e.g., ammonia, methylamine, etc. In some instances these modifications may provide sites for linking to a support or other molecule.

The peptides of the invention can be prepared in a wide variety of ways. Because of their relatively short size, the peptides (discrete epitopes or polyepitopic peptides) can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984), supra.

Alternatively, preparation of peptides of the invention can comprise use of recombinant DNA technology wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook, et al., Molecular Cloning, A Laboratory Manual, Cold Spring Harbor Press, Cold Spring Harbor, N.Y. (1982), which is incorporated herein by reference. Thus, fusion proteins which comprise one or more peptide sequences of the invention can be used to present the appropriate T-cell epitope.

As the coding sequence for peptides of the length contemplated herein can be synthesized by chemical techniques, for example, the phosphotriester method of Matteucci, et al., J. Am. Chem. Soc. 103:3185 (1981), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences.

The peptides of the present invention and pharmaceutical and vaccine compositions thereof are useful for administration to mammals, particularly humans, to therapeutically treat and/or prevent infections and cancer. Examples of diseases which can be treated using the immunogenic peptides of the invention include prostate cancer, hepatitis B, hepatitis C, AIDS, renal carcinoma, cervical carcinoma, lymphoma, CMV infection and condlyloma acuminatum.

For pharmaceutical compositions, the immunogenic peptides of the invention are often administered to an individual already suffering from cancer or infected with the virus of interest. Those in the incubation phase or the acute phase of infection can be treated with the immunogenic peptides separately or in conjunction with other treatments, as appropriate. In therapeutic applications, compositions are administered to a patient in an amount sufficient to elicit an effective CTL response to the infectious disease agent or tumor antigen and to cure or at least partially arrest symptoms and/or complications. An amount adequate to accomplish this is defined as a “therapeutically effective dose” or “unit dose”. Amounts effective for this use will depend on, e.g., the peptide composition, the manner of administration, the stage and severity of the disease being treated, the weight and general state of health of the patient, and the judgment of the prescribing physician. Generally for humans the dose range for the initial immunization (that is for therapeutic or prophylactic administration) is from about 1.0 μg to about 20,000 μg of peptide for a 70 kg patient, preferably, 100 μg-, 150 μg-, 200 μg-, 250 μg-, 300 μg-, 400 μg-, or 500 μg-20,000 μg, followed by boosting dosages in the same dose range pursuant to a boosting regimen over weeks to months depending upon the patient's response and condition by measuring specific CTL activity in the patient's blood. In embodiments where recombinant nucleic acid administration is used, the administered material is titrated to achieve the appropriate therapeutic response. It must be kept in mind that the peptides and compositions of the present invention may generally be employed in serious disease states, that is, life-threatening or potentially life threatening situations. In such cases, in view of the minimization of extraneous substances in the compositions of the invention and, e.g., the relative nontoxic nature of the peptides, it is possible and may be felt desirable by the treating physician to administer substantial excesses of these compositions.

For therapeutic use, administration should begin at the first sign of infection or the detection or surgical removal of tumors or shortly after diagnosis in the case of acute infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, for instance, as described herein, the composition can be targeted to them, minimizing need for administration to a larger population.

The peptide compositions can also be used for the treatment of chronic infection and to stimulate the immune system to eliminate, e.g., virus-infected cells in carriers. It is important to provide an amount of immuno-potentiating peptide in a formulation and mode of administration sufficient to effectively stimulate a cytotoxic T-cell response. Thus, for treatment of chronic infection, immunizing doses followed by boosting doses at established intervals, e.g., from one to four weeks, may be required, possibly for a prolonged period of time to effectively immunize an individual. In the case of chronic infection, administration should continue until at least clinical symptoms or laboratory tests indicate that the infection has been eliminated or substantially abated and for a period thereafter.

The pharmaceutical compositions for therapeutic treatment are intended for parenteral, topical, oral or local administration. Peptides of the invention can be administered in a form of nucleic acids that encode the peptides. Preferably, the pharmaceutical compositions are administered parenterally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of CTL stimulatory peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected. A human unit dose form of the peptide composition is typically included in a pharmaceutical composition that comprises a human unit dose of an acceptable carrier, preferably an aqueous carrier, and is administered in a volume of fluid that is known by those of skill in the art to be used for administration of such compositions to humans.

The peptides of the invention may also be administered via liposomes, which serve to target the peptides to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

Accordingly, an aspect the present invention is directed to vaccines which contain as an active ingredient an immunogenically effective amount of an immunogenic peptide as described herein. The peptides may also be administered in the form of nucleic acids which encode peptides of the invention upon expression in the recipient. The peptide(s) may be introduced into a host, including humans, linked to its own carrier or as a homopolymer or heteropolymer of active peptide units. Such a polymer has the advantage of increased immunological reaction and, where different peptides are used to make up the polymer, the additional ability to induce antibodies and/or CTLs that react with different antigenic determinants of the virus or tumor cells. Useful carriers are well known in the art, and include, e.g., thyroglobulin, albumins such as human serum albumin, tetanus toxoid, polyamino acids such as poly(lysine:glutamic acid), influenza, hepatitis B virus core protein, hepatitis B virus recombinant vaccine and the like. The vaccines can also contain a physiologically tolerable (acceptable) diluent such as water, phosphate buffered saline, or saline, and further typically include an adjuvant. Materials such as incomplete Freund's adjuvant, aluminum phosphate, aluminum hydroxide, or alum are materials well known in the art as adjuvants. And, as mentioned above, CTL responses can be primed by conjugating peptides of the invention to lipids, such as P₃CSS. Upon immunization with a peptide composition as described herein, via injection, aerosol, oral, transdermal or other route, the immune system of the host responds to the vaccine by producing large amounts of CTLs specific for the desired antigen, and the host becomes at least partially immune to later infection, or resistant to developing chronic infection.

In some instances it may be desirable to combine the peptide vaccines of the invention with vaccines which induce neutralizing antibody responses to the virus of interest, particularly to viral envelope antigens.

For therapeutic or immunization purposes, peptides of the invention can be administered in the form of nucleic acids encoding one or more of the peptides of the invention. The nucleic acids can encode a peptide of the invention and optionally one or more additional molecules. A number of methods are conveniently used to deliver nucleic acids to a patient. For instance, nucleic acid can be delivered directly, as “naked DNA”. This approach is described, for instance, in Wolff, et al., Science 247: 1465-1468 (1990) as well as U.S. Pat. Nos. 5,580,859 and 5,589,466. Nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO 96/18372; WO 93/24640; Mannino & Gould-Fogerite, BioTechniques 6(7): 682-691 (1988); Rose U.S. Pat. No. 5,279,833; WO 91/06309; and Felgner, et al., Proc. Natl. Acad. Sci. USA 84: 7413-7414 (1987).

The peptides of the invention can also be expressed by attenuated viral hosts, such as vaccinia or fowlpox. This approach involves the use of vaccinia virus as a vector to express nucleotide sequences that encode the peptides of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described, e.g., in Stover, et al. (Nature 351:456-460 (1991)). A wide variety of other vectors useful for therapeutic administration or immunization of the peptides of the invention, e.g., Salmonella typhi vectors and the like, will be apparent to those skilled in the art from the description herein.

A preferred means of administering nucleic acids encoding the peptides of the invention uses minigene constructs encoding multiple epitopes of the invention optionally together with other molecules. To create a DNA sequence encoding the selected CTL epitopes (minigene) for expression in human cells, the amino acid sequences of the epitopes are, e.g., reverse translated. A human codon usage table is used to guide the codon choice for each amino acid. These epitope-encoding DNA sequences are directly adjoined, creating a molecule that encodes a continuous polypeptide sequence. Optionally, to optimize expression and/or immunogenicity, additional elements can be incorporated into the minigene design. Examples of amino acid sequence that could be reverse translated and included in the minigene sequence include: helper T lymphocyte epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. he ends of the oligonucleotides are joined using T4 DNA ligase. This synthetic minigene, encoding the CTL epitope polypeptide, can then cloned into a desired expression vector.

Standard regulatory sequences well known to those of skill in the art are generally included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immunostimulatory sequences (ISSs or CpGs) play a role in the immunogenicity of DNA vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g. LeIF) or costimulatory molecules. Moreover, if helper T lymphocyte (HTL) epitopes are employed, the HTL epitopes can be joined to intracellular targeting signals and expressed separately from the CTL epitopes. This allows direction of the HTL epitopes to a cell compartment different than the CTL epitopes. This can facilitate more efficient entry of HTL epitopes into the MHC class II pathway, thereby facilitating and improving CTL induction. In contrast to CTL induction, specifically decreasing the immune response by co-expression of immunosuppressive molecules (e.g. TGF-β may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Therapeutic quantities of plasmid DNA are produced, e.g., by fermentation in E. coli, followed by purification. Aliquots from the working cell bank are used to inoculate fermentation medium (such as Terrific Broth), and grown to saturation in shaker flasks or a bioreactor according to well known techniques. Plasmid DNA can be purified using standard bioseparation technologies such as solid phase anion-exchange resins supplied by Quiagen. If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA can be introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51Cr release, indicates production of MHC presentation of minigene-encoded CTL epitopes.

In vivo immunogenicity is a second approach for functional testing of minigene DNA formulations. Transgenic mice expressing appropriate human MHC molecules can be immunized with the DNA product. The dose and route of administration are formulation dependent (e.g. IM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Transgenic animals of appropriate haplotypes may additionally provide a useful tools in optimizing the in vivo immunogenicity of minigene DNA. In addition, animals such as monkeys having conserved HLA molecules with cross reactivity to CTL epitopes recognized by human MHC molecules can be used to determine human immunogenicity of CTL epitopes (Bertoni, et al., J. Immunol. 161:4447-4455 (1998)).

Such in vivo studies are required to address the variables crucial for vaccine development, which are not easily evaluated by in vitro assays, such as route of administration, vaccine formulation, tissue biodistribution, and involvement of primary and secondary lymphoid organs. Because of its simplicity and flexibility, HLA transgenic mice represent an attractive alternative, at least for initial vaccine development studies, compared to more cumbersome and expensive studies in higher animal species, such as nonhuman primates.

Antigenic peptides can be used to elicit CTL ex vivo, as well. The resulting CTL, can be used to treat chronic infections (e.g., viral or bacterial) or tumors in patients that do not respond to other conventional forms of therapy, or will not respond to a peptide vaccine approach of therapy. Ex vivo CTL responses to a particular pathogen (infectious agent or tumor antigen) are induced by incubating in tissue culture the patient's CTL precursor cells (CTLp) together with a source of antigen-presenting cells (APC) and the appropriate immunogenic peptide. After an appropriate incubation time (typically 1-4 weeks), in which the CTLp are activated and mature and expand into effector CTL, the cells are infused back into the patient, where they will destroy their specific target cell (an infected cell or a tumor cell).

The peptides may also find use as diagnostic reagents. For example, a peptide of the invention may be used to determine the susceptibility of a particular individual to a treatment regimen which employs the peptide or related peptides, and thus may be helpful in modifying an existing treatment protocol or in determining a prognosis for an affected individual.

For example, a peptide of the invention may be used in a tetramer staining assay to assess peripheral blood mononuclear cells for the presence of antigen-specific CTLs following exposure to a pathogen or immunogen. The HLA-tetrameric complex is used to directly visualize antigen-specific CTLs (see, e.g., Ogg, et al. Science 279:2103-2106, 1998; and Altman, et al. Science 174:94-96, 1996) and determine the frequency of the antigen-specific CTL population in a sample of peripheral blood mononuclear cells. A tetramer reagent using a peptide of the invention may be generated as follows: A peptide that binds to an allele-specific HLA molecules, or supertype molecules, is refolded in the presence of the corresponding HLA heavy chain and β₂-microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells may then be identified, for example, by flow cytometry. Such an analysis may be used for diagnostic or prognostic purposes.

In addition, the peptides may also be used to predict which individuals will be at substantial risk for developing chronic infection.

The present application is related to U.S. Ser. No. 08/589,108, filed Jan. 23, 1996 and now abandoned, and to U.S. Ser. No. 08/205,713 filed Mar. 4, 1994, which is a continuation-in-part of U.S. Ser. No. 08/159,184, filed Nov. 29, 1993 and now abandoned, which is a continuation-in-part of U.S. Ser. No. 08/073,205 filed Jun. 4, 1993 and now abandoned, which is a continuation-in-part of U.S. Ser. No. 08/027,146 filed Mar. 5, 1993 and now abandoned. The application is also related to U.S. Serial No. 60/013,980 filed Mar. 21, 1996 and now abandoned, U.S. Ser. No. 08/454,033 filed May 26, 1995, U.S. Ser. No. 08/349,177 filed Dec. 2, 1994, and U.S. Ser. No. 08/753,622 filed Jan. 27, 1996 and now abandoned. Each of the above-referenced applications is incorporated herein by reference.

### EXAMPLES

### Example 1

### Peptides

Peptides utilized were synthesized as previously described by Ruppert, J. et al., “Prominent Role of Secondary Anchor Residues in Peptide Binding to HLA-A2.1 Molecules,” Cell 74:929-937 (1993) or purchased as crude material from Chiron Mimotopes (Chiron Corp., Australia). Synthesized peptides were typically purified to >95% homogeneity by reverse phase HPLC. Purity of synthesized peptides was determined using analytical reverse-phase HPLC and amino acid analysis, sequencing, and/or mass spectrometry. Lyophilized peptides were resuspended at 4-20 mg/ml in 100% DMSO, then diluted to required concentrations in PBS+0.05% (v/v) NP40 (Fluka Biochemika, Buchs, Switzerland).

### Example 2

### MHC Purification

The EBV transformed cell lines JY (A*0201), M7B (A*0202), FUN (A*0203), DAH (A*0205), CLA (A*0206), KNE (A*0207), AP (A*0207), and AMAI (A*6802) were used as the primary source of MHC molecules. Single MHC allele transfected 721.221 lines were also used as sources of A*0202 and A*0207. Cells were maintained in vitro by culture in RPMI 1640 medium (Flow Laboratories, McLean, Va.), supplemented with 2 mM L-glutamine (GIBCO, Grand Island, N.Y.), 100 U (100 μg/ml) penicillin-streptomycin solution (GIBCO), and 10% heat-inactivated FCS (Hazelton Biologics). Large scale cultures were maintained in roller bottles.

HLA molecules were purified from cell lysates (Sidney, J., et al., “The Measurement of MHC/Peptide Interactions by Gel Infiltration,” Curr Prot Immunol 18.3.1-18.3.19 (1998)). Briefly, cells were lysed at a concentration of 10⁸ cells/ml in 50 mM Tris-HCL, pH 8.5, containing 1% (v/v) NP-40 150 mM NaCl, 5 mM EDTA, and 2 mM PMSF. Lysates were then passed through 0.45 μM filters, cleared of nuclei and debris by centrifugation at 10,000×g for 20 minutes and MHC molecules purified by monoclonal antibody-based affinity chromatography.

For affinity purification, columns of inactivated Sepharose CL4B and Protein A Sepharose were used as pre-columns. Class I molecules were captured by repeated passage over Protein A Sepharose beads conjugated with the anti-HLA (A, B, C) antibody W6/32 (Sidney, J., et al., supra). HLA-A molecules were further purified from HLA-B and -C molecules by passage over a B1.23.2 column. After 2 to 4 passages the W6/32 column was washed with 10-column volumes of 10 mM Tris-HCL, pH 8.0 with 1% (v/v) NP-40, 2-column volumes of PBS, and 2-column volumes of PBS containing 0.4% (w/v) n-octylglucoside. Class I molecules were eluted with 50 mM dimethylamine in 0.15 M NaCl containing 0.4% (w/v) n-octylglucoside, pH 11.5.A 1/26 volume of 2.0 M Tris, pH 6.8, was added to the eluate to reduce the pH to ˜8.0. The eluate was then concentrated by centrifugation in Centriprep 30 concentrators at 2000 rpm (Amicon, Beverly, Mass.). Protein purity, concentration, and effectiveness of depletion steps were monitored by SDS-PAGE and BCA assay.

### Example 3

### MHC-Peptide Binding Assays

Quantitative assays to measure the binding of peptides to soluble Class I molecules are based on the inhibition of binding of a radiolabeled standard peptide. These assays were performed as previously described (Sidney, J., et al., supra.). Briefly, 1-10 nM of radiolabeled peptide was co-incubated at room temperature with 1 μM to 1 nM of purified MHC in the presence of 1 μM human β₂-microglubulin (Scripps Laboratories, San Diego, Calif.) and a cocktail of protease inhibitors. Following a two day incubation, the percent of MHC bound radioactivity was determined by size exclusion gel filtration chromatography using a TSK 2000 column. Alternatively, the percent of MHC bound radioactivity was determined by capturing MHC/peptide complexes on W6/32 antibody coated plates, and determining bound cpm using the TopCount microscintillation counter (Packard Instrument Co., Meriden, Conn.) (Southwood, et al., Epimmune Technical Report Epi 063-99).

The radio labeled standard peptide utilized for the A*0201, A*0202, A*0203, A*0205, A*0206, and A*0207 assays was an F₆>Y analog of the HBV core 18-27 epitope (sequence FLPSDYFPSV). The average IC₅₀ of this peptide for each molecule was 5.0, 4.3, 10, 4.3, 3.7, and 23 nM, respectively. A C₄>A analog of HBV pol 646 (sequence FTQAGYPAL), or MAGE 1 282 (sequence YVIKVSARV), was utilized as the label for the A*6802 assay. Their IC₅₀s for A*6802 were 40 and 8 nM, respectively.

In the case of competitive assays, the concentration of peptide yielding 50% inhibition of the binding of the radiolabeled peptide was calculated. Peptides were initially tested at one or two high doses. The IC₅₀ of peptides yielding positive inhibition were then determined in subsequent experiments, in which two to six further dilutions were tested. Under the conditions utilized, where [label]<[MHC] and IC₅₀ ≧[MHC], the measured IC₅₀ values are reasonable approximations of the true Kd values. Each competitor peptide was tested in two to four independent experiments. As a positive control, the unlabeled version of the radiolabeled probe was also tested in each experiment.

### Example 4

### Alternative Binding Assay

Epstein-Barr virus (EBV)-transformed homozygous cell lines, fibroblasts, CIR, or 721.22 transfectants were used as sources of HLA class I molecules. These cells were maintained in vitro by culture in RPMI 1640 medium supplemented with 2 mM L-glutamine (GIBCO, Grand Island, N.Y.), 50 μM 2-ME, 1001 g/ml of streptomycin, 100 U/ml of penicillin (Irvine Scientific) and 10% heat-inactivated FCS (Irvine Scientific, Santa Ana, Calif.). Cells were grown in 225-cm² tissue culture flasks or, for large-scale cultures, in roller bottle apparatuses. Cells were harvested by centrifugation at 1500 RPM using an IEC-CRU5000 centrifuge with a 259 rotor and washed three times with phosphate-buffered saline (PBS)(0.01 M PO₄, 0.154 M NaCl, pH 7.2).

Cells were pelleted and stored at −70° C. or treated with detergent lysing solution to prepare detergent lysates. Cell lysates were prepared by the addition of stock detergent solution [1% NP-40 (Sigma) or Renex 30 (Accurate Chem. Sci. Corp., Westbury, N.Y. 11590), 150 mM NaCl, 50 mM Tris, pH 8.0] to the cell pellets (previously counted) at a ratio of 50-100×10⁶ cells per ml detergent solution. A cocktail of protease inhibitors was added to the premeasured volume of stock detergent solution immediately prior to the addition to the cell pellet. Addition of the protease inhibitor cocktail produced final concentrations of the following: phenylmethylsulfonyl fluoride (PMSF), 2 mM; aprotinin, 5 μg/ml; leupeptin, 10 μg/ml; pepstatin, 10 μg/ml; iodoacetamide, 100 μM; and EDTA, 3 ng/ml. Cell lysis was allowed to proceed at 4° C. for 1 hour with periodic mixing. Routinely 5-10×10⁹ cells were lysed in 50-100 ml of detergent solution. The lysate was clarified by centrifugation at 15,000×g for 30 minutes at 4° C. and subsequent passage of the supernatant fraction through a 0.2 it filter unit (Nalgene).

The HLA-A antigen purification was achieved using affinity columns prepared with mAb-conjugated Sepharose beads. For antibody production, cells were grown in RPMI with 10% FBS in large tissue culture flasks (Corning 25160-225). Antibodies were purified from clarified tissue culture medium by ammonium sulfate fractionation followed by affinity chromatography on protein-A-Sepharose (Sigma). Briefly, saturated ammonium sulfate was added slowly with stirring to the tissue culture supernatant to 45% (volume to volume) overnight at 4° C. to precipitate the immunoglobulins. The precipitated proteins were harvested by centrifugation at 10,000×g for 30 minutes. The precipitate was then dissolved in a minimum volume of PBS and transferred to dialysis tubing (Spectro/Por 2, Mol. wt. cutoff 12,000-14,000, Spectum Medical Ind.). Dialysis was against PBS (≧20 times the protein solution volume) with 4-6 changes of dialysis buffer over a 24-48 hour period at 4° C. The dialyzed protein solution was clarified by centrifugation (10,000×g for 30 minutes) and the pH of the solution adjusted to pH 8.0 with 1N NaOH. Protein-A-Sepharose (Sigma) was hydrated according to the manufacturer's instructions, and a protein-A-Sepharose column was prepared. A column of 10 ml bed volume typically binds 50-100 mg of mouse IgG.

The protein sample was loaded onto the protein-A-Sepharose column using a peristaltic pump for large loading volumes or by gravity for smaller volumes (<100 ml). The column was washed with several volumes of PBS, and the eluate was monitored at A280 in a spectrophotometer until base line was reached. The bound antibody was eluted using 0.1 M citric acid at suitable pH (adjusted to the appropriate pH with 1N NaOH). For mouse IgG-1 pH 6.5 was used for IgG2a pH 4.5 was used and for IgG2b and IgG3 pH 3.0 was used. 2 M Tris base was used to neutralize the eluate. Fractions containing the antibody (monitored by A280) were pooled, dialyzed against PBS and further concentrated using an Amicon Stirred Cell system (Amicon Model 8050 with YM30 membrane). The anti-A2 mAb, BB7.2, was useful for affinity purification.

The HLA-A antigen was purified using affinity columns prepared with mAb-conjugated Sepharose beads. The affinity columns were prepared by incubating protein-A-Sepharose beads (Sigma) with affinity-purified mAb as described above. Five to 10 mg of mAb per ml of bead is the preferred ratio. The mAb bound beads were washed with borate buffer (borate buffer: 100 mM sodium tetraborate, 154 mM NaCl, pH 8.2) until the washes show A280 at based line. Dimethyl pimelimidate (20 mM) in 200 mM triethanolamine was added to covalently crosslink the bound mAb to the protein-A-Sepharose (Schneider, et al., J. Biol. Chem. 257:10766 (1982). After incubation for 45 minutes at room temperature on a rotator, the excess crosslinking reagent was removed by washing the beads twice with 10-20 ml of 20 mM ethanolamine, pH 8.2. Between each one the slurry was placed on a rotator for 5 minutes at room temperature. The beads were washed with borate buffer and with PBS plus 0.02% sodium azide.

The cell lysate (5-10×10⁹ cell equivalents) was then slowly passed over a 5-10 ml affinity column (flow rate of 0.1-0.25 ml per minute) to allow the binding of the antigen to the immobilized antibody. After the lysate was allowed to pass through the column, the column was washed sequentially with 20 column volumes of detergent stock solution plus 0.1% sodium dodecyl sulfate, 20 column volumes of 0.5 M NaCl, 20 mM Tris, pH 8.0, and 10 column volumes of 20 mM Tris, pH 8.0. The HLA-A antigen bound to the mAb was eluted with a basic buffer solution (50 mM dimethylamine in water). As an alternative, acid solutions such as 0.15-0.25 M acetic acid were also used to elute the bound antigen. An aliquot of the eluate (1/50) was removed for protein quantification using either a colorimetric assay (BCA assay, Pierce) or by SDS-PAGE, or both. SDS-PAGE analysis was performed as described by Laemmli (Laemmli, U.K., Nature 227:680 (1970)) using known amounts of bovine serum albumin (Sigma) as a protein standard. Allele specific antibodies were used to purify the specific MHC molecule. In the case of HLA-A2, the mAb BB7.2 was used.

A detailed description of the protocol utilized to measure the binding of peptides to Class I HLA molecules has been published (Sette, et al., Mol. Immunol. 31:813, 1994; Sidney, et al., in Current Protocols in Immunology, Margulies, Ed., John Wiley & Sons, New York, Section 18.3, 1998). Briefly, purified MHC molecules (5 to 500 nM) were incubated with various unlabeled peptide inhibitors and 1-10 nM ¹²⁵I-radiolabeled probe peptides for 48 h in PBS containing 0.05% Nonidet P-40 (NP40) (or 20% w/v digitonin for H-2 IA assays) in the presence of a protease inhibitor cocktail. The final concentrations of protease inhibitors (each from CalBioChem, La Jolla, Calif.) were 1 mM PMSF, 1.3 nM 1.10 phenanthroline, 73 μM pepstatin A, 8 mM EDTA, 6 mM N-ethylmaleimide, and 200 μM N alpha-p-tosyl-L-lysine chloromethyl ketone (TLCK). All assays were performed at pH 7.0.

Following incubation, MHC-peptide complexes were separated from free peptide by gel filtration on 7.8 mm×15 cm TSK200 columns (TosoHaas 16215, Montgomeryville, Pa.), eluted at 1.2 mls/min with PBS pH 6.5 containing 0.5% NP40 and 0.1% NaN₃. The eluate from the TSK columns was passed through a Beckman 170 radioisotope detector, and radioactivity was plotted and integrated using a Hewlett-Packard 3396A integrator, and the fraction of peptide bound was determined.

Radiolabeled peptides were iodinated using the chloramine-T method. A specific radiolabeled probe peptide was utilized in each assay. Typically, in preliminary experiments, each MHC preparation was titered in the presence of fixed amounts of radiolabeled peptides to determine the concentration of HLA molecules necessary to bind 10-20% of the total radioactivity. All subsequent inhibition and direct binding assays were performed using these HLA concentrations.

Since under these conditions [label]<[HLA] and IC₅₀≧[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC₅₀ of a positive control for inhibition, i.e. the reference peptide that is included in every binding assay, by the IC₅₀ for each tested peptide (typically unlabeled versions of the radiolabeled probe peptide). For database purposes, and inter-experiment comparisons, relative binding values are compiled. These values can subsequently be converted into normalized IC₅₀ nM values by dividing the standard historical IC₅₀ of the reference peptide by the relative binding of the peptide of interest. This method of data compilation has proven to be the most accurate and consistent for comparing peptides that have been tested on different days, or with different lots of purified MHC.

For example, the standard reference peptide (or positive control) for the HLA-A2.1 binding assays described herein is the peptide having a sequence of FLPSDYFPSV, which has an average historical IC₅₀ value of 5 nM in multiple, repeated binding assays. This standard value is used to normalize reported IC₅₀ values for HLA-A2.1 binding as described herein. Thus, a relative binding value of a test HLA-A2.1 motif-bearing peptide can be converted into a normalized IC₅₀ by dividing the standard reference IC₅₀ value, i.e. 5 nM, by the relative binding value of the test HLA-A2.1 motif-bearing peptide.

### Example 5

### Sequence and Binding Analysis

Using the assay described in Example 3, a relative binding value was calculated for each peptide by dividing the IC₅₀ of the positive control for inhibition by the IC₅₀ for each tested peptide. These values can subsequently be converted back into IC₅₀ nM values by dividing the IC₅₀ nM of the positive controls for inhibition by the relative binding of the peptide of interest. This method of data compilation has proved to be accurate and consistent for comparing peptides that have been tested on different days or with different lots of purified MHC. Standardized relative binding values also allow the calculation a geometric mean, or average relative binding value (ARB), for all peptides with a particular characteristic (Ruppert, J., et al., “Prominent Role of Secondary Anchor Residues in Peptide Binding to HLA-A2.1 Molecules,” Cell 74:929-937 (1993); Sidney, J., et al., “Definition of an HLA-A3-Like Supermotif Demonstrates the Overlapping Peptide Binding Repertoires of Common HLA Molecules,” Hum Immunol. 45:79-93 (1996); Sidney, J., et al., “Specificity and Degeneracy in Peptide Binding to HLA-B7-Like Class I Molecules,” J. Immunol. 157:3480-3490 (1996); Kondo, A., et al., “Prominent Roles of Secondary Anchor Residues in Peptide Binding to HLA-A24 Human Class I Molecules,” J. Immunol. 155:4307-4312 (1995); Kondo, A., et al., “Two Distinct HLA-A*0101-Specific Submotifs Illustrate Alternative Peptide Binding Modes,” Immunogenetics 45:249-258 (1997); Gulukota, K., et al., “Two Complementary Methods for Predicting Peptides Binding Major Histocompatibility Complex Molecules,” J. Mol. Biol. 267:1258-1267 (1997); Southwood, S., et al., “Several Common HLA-DR Types Share Largely Overlapping Peptide Binding Repertoires,” J. Immunol 160:3363-3373 (1998)).

Maps of secondary interactions influencing peptide binding to HLA-A2 supertype molecules based on ARB were derived as previously described (Ruppert, J. et al., “Prominent Role of Secondary Anchor Residues in Peptide Binding to HLA-A2.1 Molecules,” Cell 74:929-937 (1993); Sidney, J., et al., “Definition of an HLA-A3-Like Supermotif Demonstrates the Overlapping Peptide Binding Repertoires of Common HLA Molecules,” Hum Immunol. 45:79-93 (1996); Sidney, J., et al., “Specificity and Degeneracy in Peptide Binding to HLA-B7-Like Class I Molecules,” J. Immunol. 157:3480-3490 (1996); Kondo, A., et al., “Prominent Roles of Secondary Anchor Residues in Peptide Binding to HLA-A24 Human Class I Molecules,” J. Immunol. 155:4307-4312 (1995); Kondo, A., et al., “Two Distinct HLA-A*0101-Specific Submotifs Illustrate Alternative Peptide Binding Modes,” Immunogenetics 45:249-258 (1997); Gulukota, K., et al., “Two Complementary Methods for Predicting Peptides Binding Major Histocompatibility Complex Molecules,” J. Mol. Biol. 267:1258-1267 (1997)). Essentially, all peptides of a given size (8, 9, 10 or 11 amino acids) and with at least one tolerated main anchor residue were selected for analysis. The binding capacity of peptides in each size group was analyzed by determining the ARB values for peptides that contain specific amino acid residues in specific positions. For determination of the specificity at main anchor positions ARB values were standardized relative to the ARB of peptides carrying the residue associated with the best binding. For secondary anchor determinations, ARB values were standardized relative to the ARB of the whole peptide set considered. That is, for example, an ARB value was determined for all 9-mer peptides that contain A in position 1, or F in position 7, etc. Because of the rare occurrence of certain amino acids, for some analyses residues were grouped according to individual chemical similarities as previously described (Ruppert, J. et al., supra; Sidney, J., et al., supra; Sidney, J., et al., supra; Kondo, A., et al., supra; Kondo, A., et al., supra; Gulukota, K., et al., supra; Southwood, S., et al., supra).

Frequencies of HLA-A2-Supertype Molecules

To select a panel of A2-supertype molecules representative of the allelic forms most frequent in major ethnic groups, unpublished population typing data from D. Mann and M. Fernandez-Vina were utilized. These data were consistent with published data (Sudo, T., et al., “DNA Typing for HLA Class I Alleles: I. Subsets of HLA-A2 and of -A28,” Hum. Immunol. 33:163-173 (1992); Ellis, J. M., et al., “Frequencies of HLA-A2 alleles in Five US Population Groups,” Hum. Immunol. 61:334-340 (2000); Krausa, P., et al., “Genetic Polymorphism Within HLA-A*02: Significant Allelic Variation Revealed in Different Populations,” Tissue Antigens 45:233-231 (1995) and Imanishi, T., et al., “Allele and Haplotype Frequencies for HLA and Complement Loci in Various Ethnic Groups” Tsuji, K., et al., (eds): HLA 1991, Proceedings of the Eleventh International Histo-Compatibility Workshop and Conference, Vol. 1., Oxford University Press, Oxford, pp. 1065-1220 (1992)), and are shown in Table 3. For the four major ethnic groups considered, it was apparent that seven HLA alleles represent the vast majority of A2 supertype alleles. Included in this group are A*0201, A*0202, A*0203, A*0205, A*0206, A*0207, and A*6802. Each of these alleles is present in 2% or more of the general population, and also occur with a frequency greater than 5% in at least one major ethnicity. Other alleles are represented with only minor frequencies of 1.3%, or less, in any one major ethnic group. Furthermore, none of the minor alleles are present with a frequency greater than 1% in the general population. Based on these observations, A*0201, A*0202, A*0203, A*0205, A*0206, A*0207, and A*6802 were selected for studies defining peptide binding specificity and cross-reactivity in the A2-supertype.

Main Anchor Positions of A2 Supertype Molecules

Previous studies indicated a largely overlapping peptide binding specificity for a set of Class I molecules designated as the A2-supertype. Here, the main peptide binding specificity of A2-supertype molecules was examined in more detail. Some of these results have been published previously, and are shown here only for reference purposes (Rupert, J., et al., supra and Sidney, J., et al., “The HLA-A*0207 Peptide Binding Repertoire is Limited to a Subset of the A*0201 Repetoire,” Hum. Immunol., 58:12-20 (1997)).

In a first series of studies, non-conservative lysine (K) substitutions were introduced at every position of two peptides previously noted to bind multiple A2-supertype molecules: 1) the HCV NS3 590 9-mer peptide (sequence YLVAYQATV), and 2) the HBV core 18 F₆>Y 10-mer analog peptide (sequence FLPSDYFPSV). These peptides were tested for their capacity to bind A*0201, A*0202, A*0203, A*0205, A*0206, A*0207 and A*6802. In the case of the HCV NS3 590 peptide (Table 4a), K substitutions at position 2 and the C-terminus resulted in greater than 100-fold reduction in binding to each HLA molecule. Greater than 100-fold decreases in binding were also noted in the context of A*6802 when K was substituted in positions 1 and 5. Reductions in binding capacity in the 10-100-fold range were noted when substitutions were made at several other positions, notably positions 3 and 7. When the 10-mer HBV core 18 F₆>Y ligand (Table 4b) was investigated, greater than 100-fold reductions in binding capacity were again noted when the peptide was substituted at position 2 and the C-terminus. Significant reductions in binding were also observed following substitution at position 7.

Together, these data suggest that A2-supertype molecules bind both 9- and 10-mer peptide ligands via anchor residues in position 2 and at the C-terminus. The presence of an additional primary or secondary anchor towards the middle of the peptide is demonstrated by the fact that the binding of both the 9-mer and 10-mer peptides was usually reduced by substitutions at position 7.

Specificity of the Position 2 and C-Terminal Anchor Residues

Based on these results, the ligand specificity of A2-supertype molecules at position 2 and the C-terminus was analyzed using additional HCV NS3 590 and HBV core 18 F₆>Y single substitution analogs, and also single substitution analogs of a poly-alanine peptide (peptide 953.01; sequence ALAKAAAAV). For these analyses, preferred amino acids for anchor residues were defined as those associated with a binding capacity within 10-fold of the optimal residue. Amino acids whose relative binding capacity was between 0.01 and 0.1 were defined as tolerated, and those associated with a binding capacity less than 0.01 were considered as non-tolerated.

At position 2 small aliphatic and hydrophobic residues were found to be generally tolerated, while other residues, including large polar, aromatic, and charged residues were typically not well tolerated (Tables 5a-c). L, I, V, and M were preferred as anchor residues in most (>80%) contexts. A, T, Q, and S were less frequently preferred as anchor residues, but were either preferred or tolerated in >80% of the contexts examined (Table 5d). None of the other amino acids examined were preferred in any context and only rarely tolerated.

At the C-terminus (Tables 6a-c), V was found to be the optimal residue in the context of all 3 parent peptides for A*0201, A*0206, and A*6802, and in 2 out of 3 cases for A*0203 and A*0205. Overall, either V or L was the optimal C-terminal residue for each molecule, regardless of the peptide tested. The aliphatic/hydrophobic amino acids V, L, and I were preferred as anchor residues in >66.7% of the MHC-peptide contexts (Table 6d). M, A, and T were tolerated approximately 50% of the time. Other residues examined were either not accepted at all, or were tolerated only rarely.

A Re-Evaluation of the Peptide Binding Specificity of A*0201

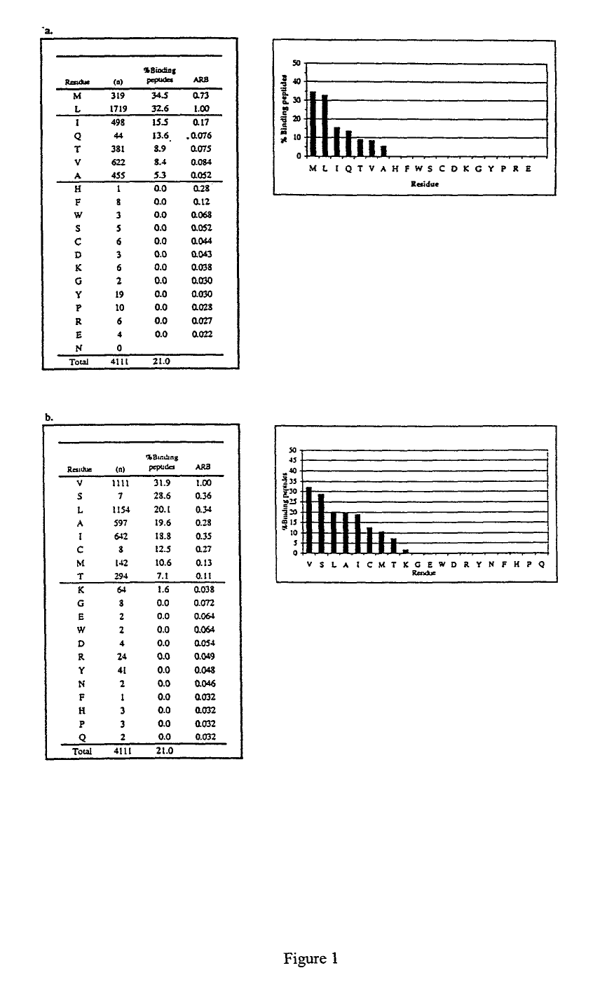

The fine specificity of A*0201 binding was investigated in more detail using a database of over 4000 peptides between 8- and 11-residues in length. It was found that over 30% of the peptides bearing L or M in position 2 bound A*0201 with affinities of 500 nM, or better (FIG. 1a). Between 5 and 15% of the peptides bearing the aliphatic residues I, V, A, T, and Q bound with IC₅₀s of 500 nM, or better. No other residue, including aromatic (F, W, and Y), charged (R, H, K, D, and E), polar (S and N) and small (C, G, and P) residues, was associated with IC₅₀s of 500 nM, or better.

Consistent with the single substitution analysis, V was found to be the optimal A*0201 C-terminal anchor residue (FIG. 1b). Overall, 31.9% of the peptides with V at the C-terminus were A*0201 binders. I, L, S, C, M, T and A were also tolerated, with 7.1 to 28.6% of the peptides binding with an IC₅₀ of 500 nM, or better.

The correlation between peptide length (between 8 and 11 residues) and binding capacity was analyzed next. It was found that 27.6% of the 9-mer peptides bound with IC₅₀ of 500 nM, or less, in good agreement with previous estimates (Rupert, J., et al., supra) (Table 7a).

Longer peptides were also capable of binding, although somewhat less well; 17.8% of 10-mer, and 14.5% of the 11-merpeptides had affinities of 500 nM or better. Finally, it was noted that 8-mer peptides bound A*0201 only rarely, with 3.5% of the peptides having binding capacities better than 500 nM.

The A*0201 peptide binding database was further analyzed to assess the stringency of the A*0201 motif. As expected, peptides with preferred residues in each anchor position bound most frequently (48.7%), and with higher average relative binding capacity than other peptides in the library (Table 7b). Peptides with one preferred residue and one tolerated residue also bound relatively frequently, in the 17.6 to 28.4% range. Finally, peptides with at least one non-tolerated residue, or with tolerated residues at both main anchor positions, bound only rarely, if at all, with frequencies of binding in the 0-7.1% range. No significant difference was detected in terms of primary anchor preferences as a function of ligand size.

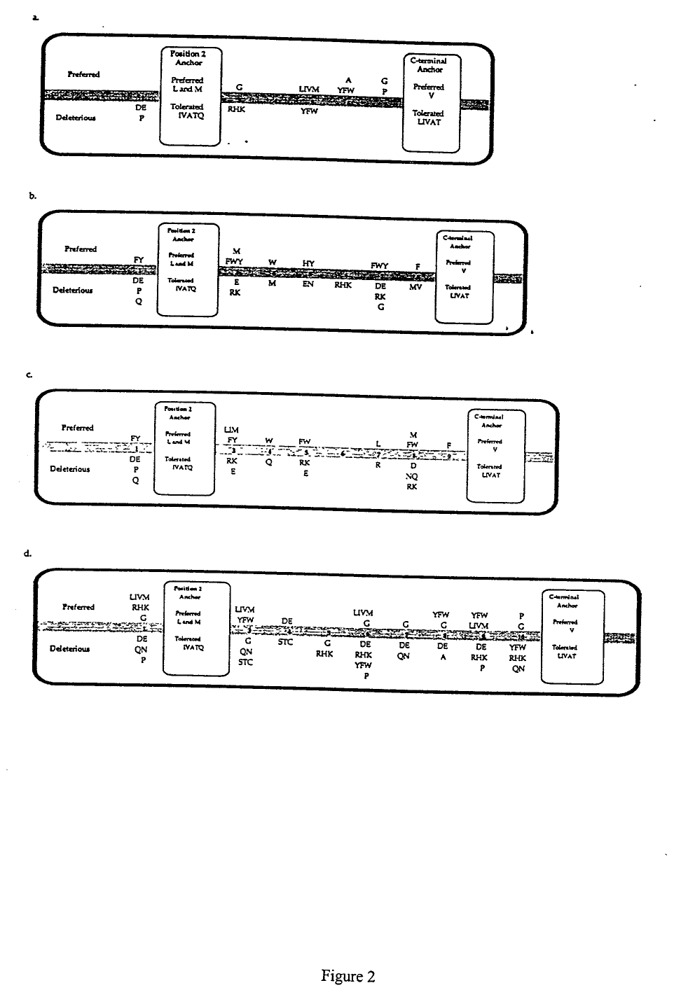

To identify secondary anchor effects, the binding capacity of peptides in each size group was further analyzed by determining the ARB values for peptides that contain a particular amino acid residues in specific, but size dependent, position. The resulting ARB values, by corresponding residue/position pairs, for 8-11-mer sequences are shown in Table 8a-d. Summary maps are shown in FIGS. 2a-d. In most positions, some secondary influence could be detected. The majority (55%) of the negative influences involved the presence of acidic (D and E) or basic (R, H, and K) residues. Proline (P) and large polar residues (Q, and N) were also frequently disruptive. While each particular size was associated with unique preferences, in most instances (79%) preferred residues were aromatic (F, W, or Y) or hydrophobic (L, I, V, or M). Most peptide lengths exhibited a preference for F, Y and M in position 3. Similarly, all peptide sizes shared a preference for aromatic or hydrophobic residues in the C-2 position. Several distinct preference patterns were also observed for peptides of a given size. For example, 8-mer peptides did not have any preference in either position 1 or position 3 for the hydrophobic or aromatic residues preferred by 9-, 10-, and 11-mer peptides. 11-mer peptides were unique in the preference for G in multiple positions throughout the middle of the peptide.

Main Anchor Specificities of Other A2-Supertype Molecules

In the next set of analyses, the main anchor specificities of A*0202, A*0203, A*0206, and A*6802, four of the most prevalent A2-supertype alleles next to A*0201, was assessed. Peptides in the A2-supertype binding database often reflect selection using an A*0201-based bias, such as the selection of only A*0201 binding peptides, or the selection of peptides scoring high in A*0201 algorithms. As a result, in most cases, peptide binding data for non-A*0201 molecules is available for only peptides with supertype preferred and tolerated residues. Despite this limitation, a database of about 400 peptides was available for study. A database of sufficient size was not available to allow analysis of A*0205 and A*0207, although an analysis of the specificity of A*0207 has been published previously (Sidney, J., et. al., supra).

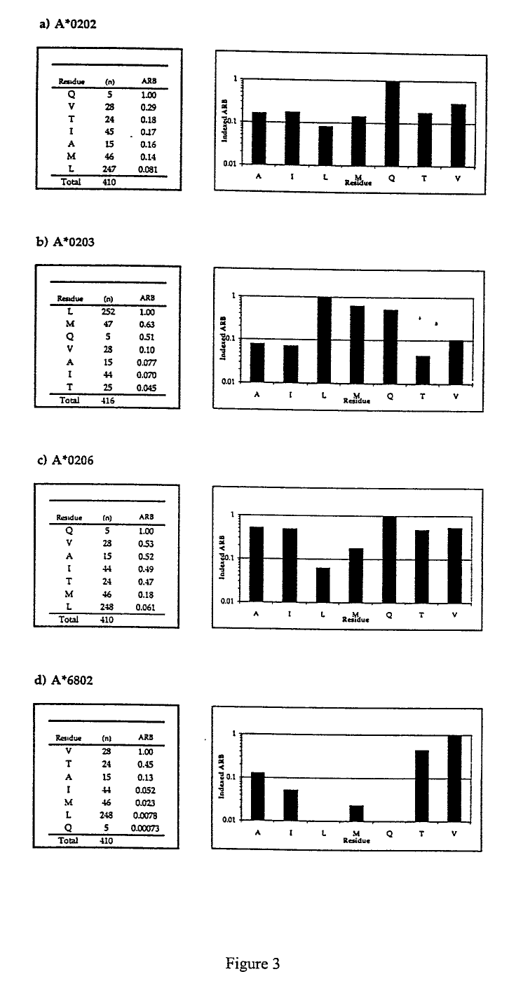

Analyses of the position 2 specificities are summarized in FIGS. 3a-d. In general, V, T, A, I, and M were tolerated in the context of each molecule. Allele specific preferences were also noted. In the case of A*0202 Q was the most preferred residue. Other residues (L, I, V, A, T and M) were tolerated, and were roughly equivalent, with ARB in the 0.08-0.30 range. By contrast, A*0203 had a preference for L, M and Q. Residues V, A, I and T were associated with lower overall binding affinities. A third pattern was noted for A*0206, where Q, V, I, A, and T were all well tolerated with ARB values between 0.47 and 1.0, while L and M were less well tolerated. Finally, for A*6802 V and T were the optimal residues, with ARB >0.45. A was also preferred, but with a lower ARB (0.13). Significant decreases in binding were seen with I and M, which had ARB between 0.050 and 0.020. L and Q were not tolerated, with ARB <0.010.

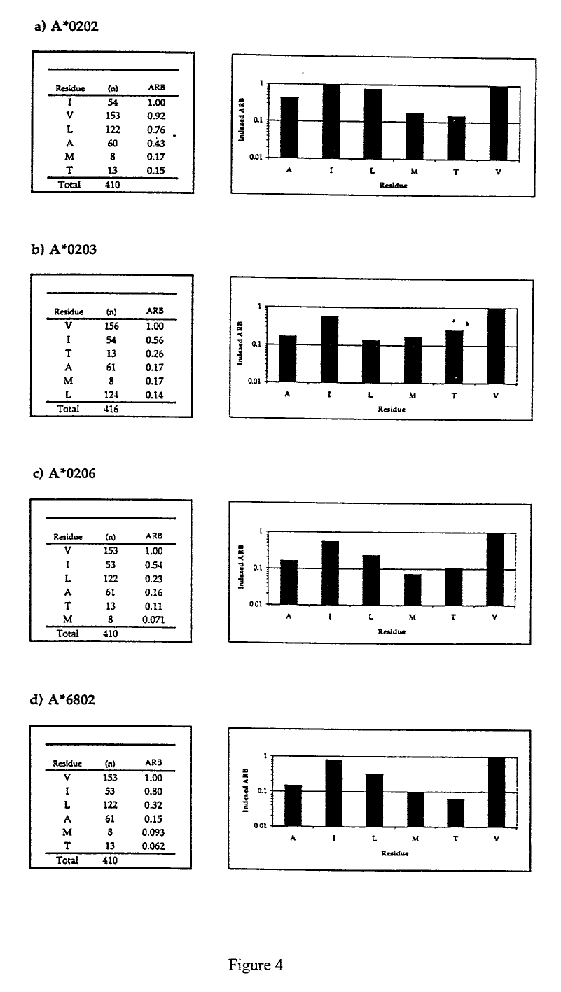

At the C-terminus, I, V, L, A, M and T were tolerated by all A2-supertype molecules tested, with ARB >0.060 (FIGS. 4a-d). I and V were the two residues most preferred by each allele; V was the optimal residue for A*0203, A*0206, and A*6802. L was typically the next most preferred residue. T, A, and M were usually associated with lower ARB values.

In conclusion, the position 2 and C-terminal anchor residues preferred or tolerated by A*0201 were also well tolerated by other A2-supertype molecules. While each allele had a somewhat unique pattern of preferences at position 2, the patterns of preferences exhibited by each allele at the C-terminus were fairly similar.

Secondary Influences on Peptide Binding to A2-Supertype Molecules

The same library of peptide ligands was analyzed to determine the ligand size preferences of A*0202, A*0203, A*0206, and A*6802. We found that for each molecule 9-11 mer peptides were well tolerated, with ARB >0.36 (Table 9 a-d). For A*0203, A*0206, and A*6802, 9-mer peptides were optimal, but 10-mers were optimal in the case of A*0202. For all alleles, 8-mer peptides were much less well tolerated, with ARB in each case <0.11.

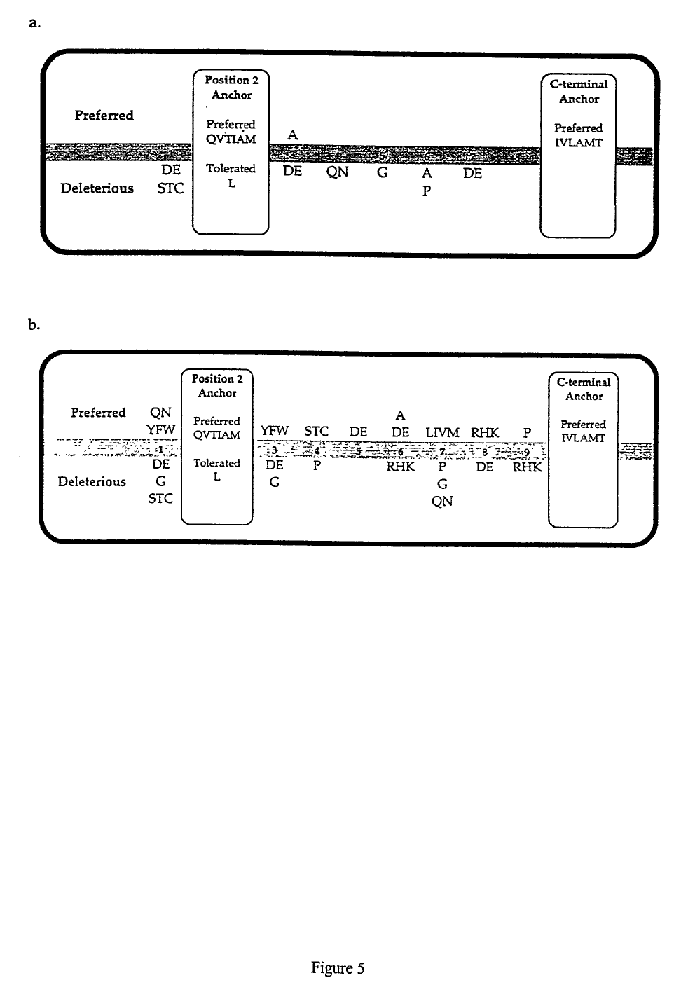

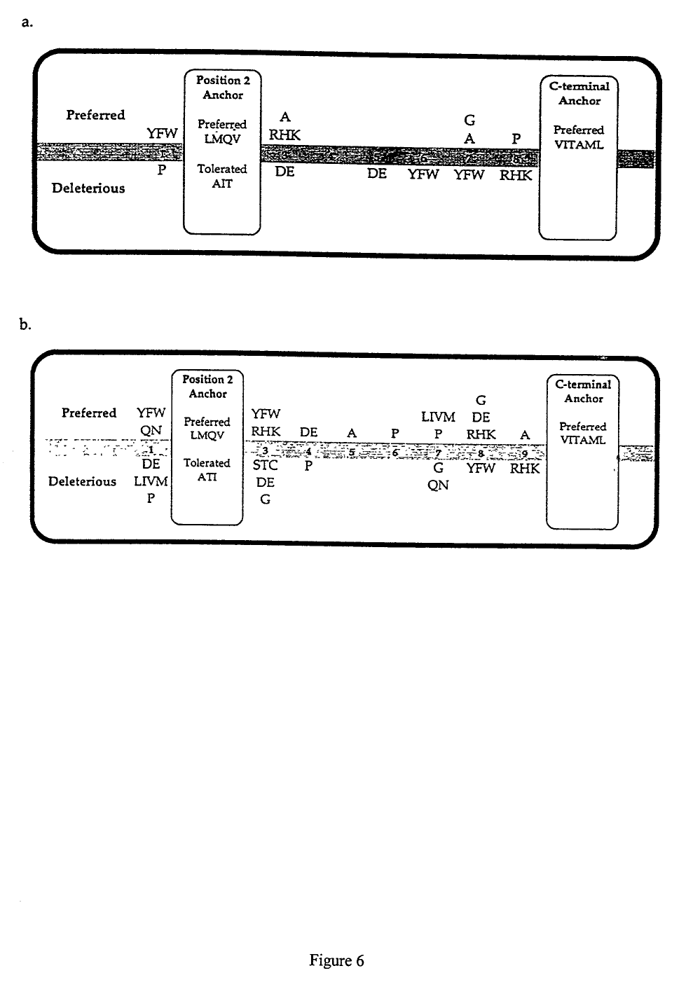

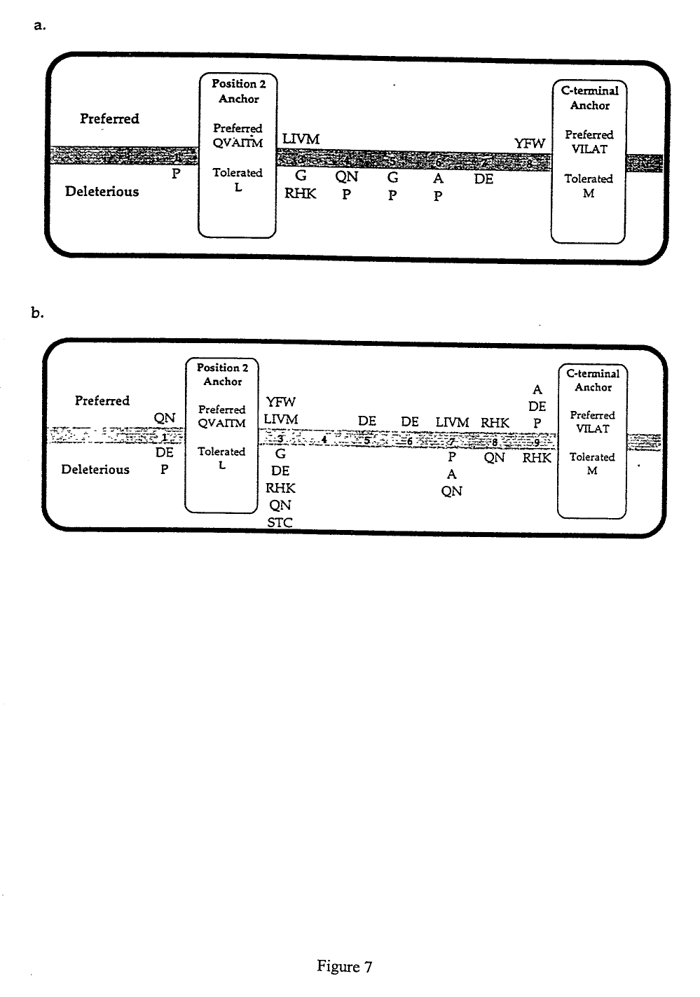

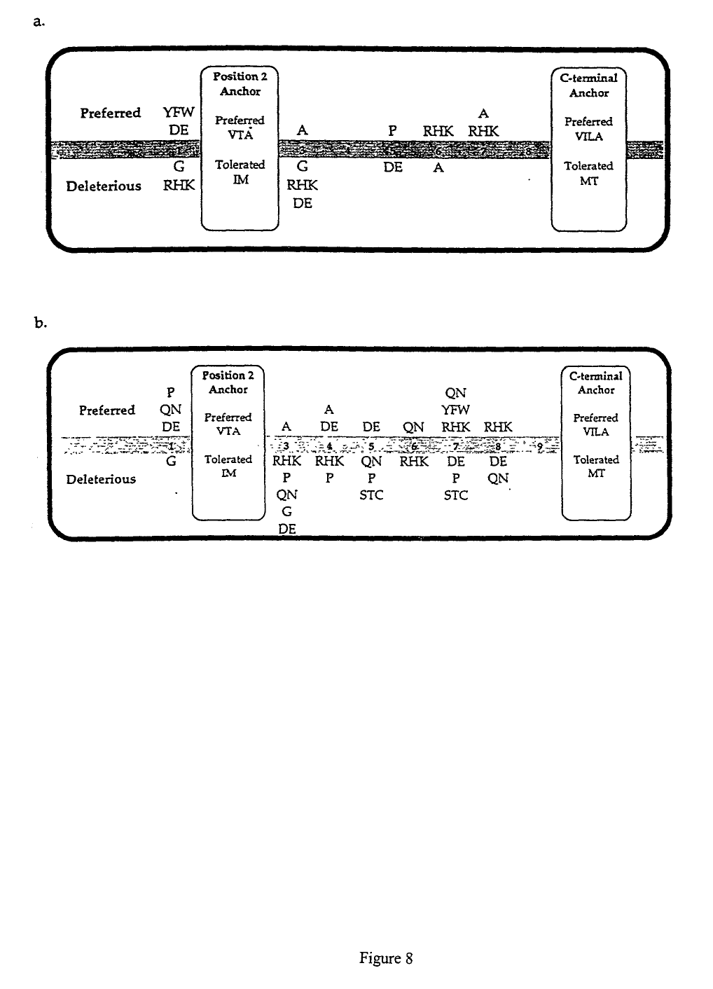

The influence of secondary anchor residues on the capacity of peptides to bind A*0202, A*0203, A*0206, and A*6802 was examined next. The number of peptides available only allowed analysis of 9- and 10-mer ligands. The ARB values for 9-mer and 10-mer peptides as a function of the presence of a particular residue in a specific position are shown in Tables 10-13, and summary maps in FIGS. 5-8. Deleterious effects were frequently (35%) associated with charged residues (D, E, R, H, or K). An additional 35% of the deleterious influences could be attributed to G or P. Positive influences were relatively evenly attributed to basic (R, H, K), acid (D, E), hydrophobic (F, W, Y, L, I, V, M) or small (A, P) residues.

While each molecule had a distinctive pattern of preferences and aversions, some common trends could be noted in the case of 10-mer peptides. For example, for all molecules Q and N were preferred in position 1, and R, H, and K were preferred in position 8. D, E, and G were uniformly deleterious for 10-mer peptides in position 3. Consensus preferences or aversions were not noted for 9-mer peptides.

In summary, the data in this section describe detailed motifs for 9- and 10-mer peptides binding to A*0202, A*0203, A*0206, and A*6802. Each motif is characterized by specific features associated with good, or poor, binding peptides.

A Consensus A2-Supermotif

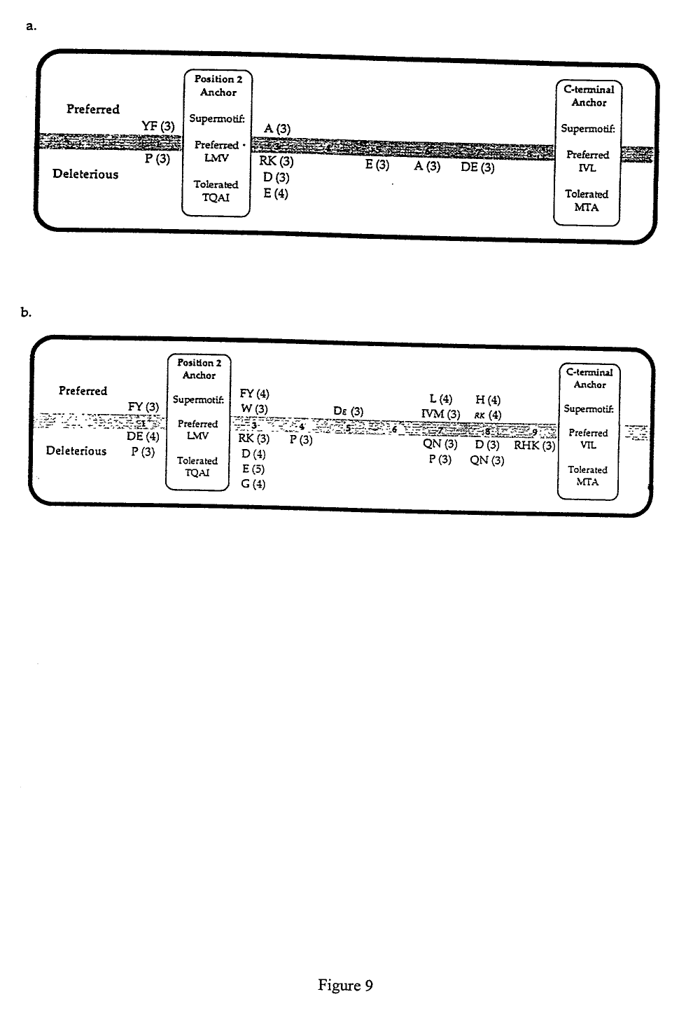

The motifs described above for A2 supertype molecules are very similar and largely overlapping. In this respect, a consensus motif can be identified that incorporates features commonly shared by the molecule-specific motifs (FIG. 9). The consensus motif specifies the presence of hydrophobic and aliphatic residues in position 2 of peptide ligands. At this position, V, L and M are preferred, while T, Q, A, and I are all tolerated. On the basis of the preference rank of each residue in the context of each A2-supertype molecule, V is the most preferred residue. At the C-terminus the consensus motif specifies the presence of hydrophobic and aliphatic residues L, I, V, M, A, and T. V is most frequently the optimal residue, while L and I are also considered preferred, typically being the next most optimal residues. M, A, and T are considered as tolerated residues.

The secondary anchor maps for A*0201, A*0202, A*0203, A*0206, and A*6802 were utilized to derive a supertype consensus secondary anchor motif for 9- and 10-mer peptides (FIG. 9). Residues considered as preferred for 3 or more A2-supertype molecules, without being deleterious for any molecule, were considered as preferred for the supertype consensus motif. Conversely, residues identified as deleterious for 3 or more molecules were designated as deleterious in the consensus motif. The consensus motif overlaps significantly with the detailed A*0201 motif, and includes a preference for aromatic residues in position 1 and/or 3, and a shared aversion for charged residues in position 3.

Correlation Between A*0201 Binding Affinity and A2-Supertype Cross-Reactivity

How well A*0201 binders also bound to other A2-supertype molecules was assessed next. It was found that peptides that bound A*0201 with good affinity (IC₅₀<500 nM) frequently bound other A2-supertype molecules (Table 14a). Between 36.1 and 73.6% of A*0201 binding peptides bound other A2-supertype molecules. Analysis of A2-supertype degeneracy as a function of A*0201 affinity also yielded interesting results. 72.8% of the peptides that bound A*0201 with IC₅₀<500 nM bound 3 or more A2-supertype molecules (Table 14b). As a general rule, the higher the binding affinity of a peptide for A*0201, the higher the likelihood that the peptide would also bind 3 or more supertype molecules. Over 96% of the peptides that bound A*0201 with affinities of 20 nM or better also bound 3 or more A2-supertype molecules. By contrast, A2-supermotif peptides that did not bind A*0201 with affinities better than 500 nM only rarely (10%) bound 3 or more A2 supermotif molecules, and never bound 4 or more molecules. In summary, this analysis of the cross-reactive binding of peptides to A*0201 and other A2-supertype molecules confirms the fact that this family of HLA molecules recognizes similar structural features in their peptide ligands.

Analysis

The results of this analysis allow for the detailed definition of the properties of peptides that bind to HLA-A*0201 and other A2-supertype molecules. The A2-supertype molecules share not only largely overlapping peptide binding specificity, but also significantly overlapping peptide binding repertoires. Specific features of peptide ligands associated with degenerate A2-supertype binding capacity were identified which provide a logical explanation for the supertype relationship.

In a previous study (6) the peptide binding specificity of A*0201 was analyzed, and a detailed motif, including the identification of secondary anchor features, was constructed. In the present analyses, performed with a 10-fold larger database, we confirmed that data and extended the analysis to include 8- and 11-mer peptides. Overall, the specificity of A*0201 for 8- and 11-mer peptides was largely similar to that for 9- and 10-mer peptides. For example, regardless of peptide size, the majority of negative influences on binding capacity were associated with the presence of charged residues in secondary anchor positions, while the majority of positive influences were associated with the presence of hydrophobic residues. The definition of detailed motifs for 8- and 11-mer peptides should allow for a more complete identification of epitopes.

Identification of A*0201 binders has been greatly facilitated by the use of the algorithms based on ARB values. In the present analyses a substantially larger database was used than previously available, allowing for a refinement of algorithm coefficients. Because the newer coefficients are based on a significantly larger data set, they are statistically more accurate and should afford more efficient and precise prediction of epitopes. Indeed, recent analysis has shown that a revised A*0201 9-mer polynomial algorithm based on a larger data set is more accurate than both an older algorithm based on a small data set, and neural network prediction methodologies.

In addition to increasing the accuracy of epitope prediction (Rupert, J., et al., supra; Sidney, J., et al., supra; Kondo, A., et al., supra; Gulukota, K., et al., supra; Parker, K. C., et al., “Sequence Motifs Important for Peptide Binding to the Human MHC Class I Molecule, HLA-A2,” J. Immunol. 149:3580-3587 (1992) and Milik, M., et al., “Application of an Artificial Neural Network to Predict Specific Class I MHC Binding Peptide Sequences,” Nature (Biotech) 16:753-756 (1998)), detailed peptide binding motifs defining both primary and secondary anchor positions allow for the rational design of optimized ligands. For example, natural sequences carrying sub-optimal residues at primary and/or secondary positions can be identified. The sub-optimal residues may be replaced with optimal anchors, generating epitopes with increased binding affinity (Sidney, J., et al., supra; Pogue, R. R., et al., “Amino-Terminal Alteration of the HLA-A*0201-Restricted Human Immunodeficiency Virus Pol Peptide Increases Complex Stability and in Vitro Immunogenicity,” Proc. Nat'l. Acad. Sci., USA, 92:8166-8170 (1995) and Bakker, A. B., et al., “Analogues of CTL epitopes With Improved MHC Class-I Binding Capacity Elicit Anti-Melanoma CTL Recognizing the Wide-Type Epitope,” Int. J. Cancer, 70:302-309 (1997)). Following this type of modification, wild type peptides that were unable to elicit responses, or were poor immunogens, may become highly immunogenic Pogue, R. R., et al., supra; Bakker, A. B., et al., supra; Parkhurst, M. R., “Improved Induction of Melanoma-Reactive CTL With Peptides From the Melanoma Antigen gp100 Modified at HLA-A*0201-Binding Peptides,” J. Immunol. 157:2539-2548 (1996); Rosenberg, S. A., et al., “Immunologic and Therapeutic Evaluation of a Synthetic Peptide Vaccine for the Treatment of Patients With Metastatic Melanoma,” Nature (Med) 4:321-327 (1998); Sarobe, P., et al., “Enhanced in vitro Potency and in vivo Immunogenicity of a CTL Epitope From Hepatitis C Virus Core Protein Following Amino Acid Replacement at Secondary HLA-A2.1 binding positions,” J. Clin. Invest. 102:1239-1248 (1998) and Ahlers, J. D., et al., “Enhanced Immunogenicity of HIV-1 Vaccine Construct by Modification of the Native Peptide Sequence,” Proc. Nat'l Acad. Sci., USA, 94:10856-10861 (1997)). The CTL induced by such analog peptides have been shown to be capable, in most instances, of recognizing target cells expressing wild type antigen sequences. This phenomenon is likely to reflect less stringent epitope binding requirements for target cell recognition compared to that needed for stimulation of naive T-cells to induce differentiation into effectors (Cho, B. K., et al., “Functional Differences Between Memory and Naive CD8 T Cells,” Proc. Nat'l. Acad. Sci. USA 96:2976-2981 (1999); Sykulev, Y., et al., “Evidence That A Single Peptide—MHC Complex On A Target Cell Can Elicit Acytolytic T Cell Response,” Immunity 4:565-571 (1996)). Thus, the detailed motifs described herein will facilitate not only in the identification of naturally occurring CTL epitopes, but also in the design of engineered epitopes with increased binding capacity and/or immunogenic characteristics.

The peptide binding specificity for other A2-supertype molecules was also investigated using single substitution analog peptides and peptide libraries. In agreement with previous reports (del Guercio, M-F, et al., “Binding of a Peptide Antigen to Multiple HLA Alleles Allows Definition of an A2-Like Supertype,” J. Immunol. 154:685-693 (1995) and (Sidney, J., et al., “Practical, Biochemical and Evolutionary Implications of the Discovery of HLA Class I Supermotifs,” Immunol Today 17:261-266 (1996)); see also reports filed for NIH-NIAID contract NO1-AI-45241), we found that the primary anchor motifs of A2-supertype molecules were remarkably similar (Sudo, T., et al., “Differences in MHC Class I Self Peptide Repertoires Among HLA-A2 Subtypes,” J. Immunol. 155:4749-4756 (1995); Rotzschke, O., et al., “Peptide Motifs of Closely Related HLA Class I Molecules Encompass Substantial Differences,” Eur J. Immunol. 22:2453-2456 (1992); and Barouch, D., et al., “HLA-A2 Subtypes Are Functionally Distinct in Peptide Binding and Presentation,” J. Exp. Med. 182:1847-1856 (1995)). The use of peptide libraries allowed detailed characterization of the secondary anchor preferences and aversions of each molecule. It was shown that, while each A2-supertype molecule had a unique specificity, a supermotif based on consensus patterns could be identified. Because the supermotif describes features of peptide ligands that are shared amongst A2-supertype molecules, it is expected to allow the efficient identification of highly cross-reactive peptides, and indicate appropriate strategies for anchor fixing, allowing modulation of the supertype degeneracy of peptide ligands. A further result of the present analysis was the derivation of coefficients that could be utilized in algorithms for predicting peptide binding to A*0202, A*0203, A*0206, and A*6802.

HLA A*0201 is by far the most prevalent A2-supertype allele, both in the general population and within major ethnic groups. Thus, the peptide screening strategy that were utilized focuses first on the identification of A*0201 binders. It was determined that over 70% of the peptides that bind to A*0201 also bind to at least 2 additional A2-supertype molecules, and that the propensity to bind other A2-supertype alleles correlated with A*0201 binding affinity.

In conclusion, the data described herein provide formal demonstration of the shared peptide binding specificity of a group of HLA-A molecules designated as the A2-supertype. Not only do these molecules recognize similar features at primary and secondary anchor positions of their peptide ligands, they also share largely overlapping peptide binding repertoires. The demonstration that these molecules share largely overlapping repertoires has a significant implication for the design of potential vaccine constructs. Indeed, the concept that A2-supertype cross-reactivity at the peptide binding level may be of immunological relevance has been demonstrated in a number of studies, in both infectious disease (Khanna R., et al., “Identification of Cytotoxic T-Cell Epitopes Within Epstein-Barr Virus (EBV) Oncogene Latent Membrane Protein 1 (LMP1): Evidence for HLA A2 Supertype-Restricted Immune Recognition of EBV-Infected Cells by LMP1-Specific Cytotoxic T lymphocytes,” Eur J Immunol, 28:451-458 (1998); Bertoletti, A., et al., “Molecular Features of the Hepatitis B Virus Nucleocapsid T-Cell Epitope 18-27: Interaction With HLA An T-Cell Receptor,” Hepatology 26:1027-1034 (1997); Livingston, B. D., et al., “Immunization With the HBV Core 18-27 Epitope Elicits CTL Responses in Humans Expressing Different HLA-A2 Supertype Molecules,” Hum Immunol 60:1013-1017, (1999); Bertoni, R., et al., “Human Histocompatibility Leukocyte Antigen-Binding Supermotifs Predict Broadly Cross-Reactive Cytotoxic T Lymphocyte Responses in Patients With Acute Hepatitis,” J Clin Invest 100:503-513 (1997); and Doolan, D. L., et al., “Degenerate Cytotoxic T-Cell Epitopes from P. falciparum Restricted by Multiple HLA-A and HLA-B Supertype Alleles,” Immunity 7:97-112 (1997)) and cancer (Fleischhauer, K., et al., “Multiple HLA-A Alleles Can Present an Immunodominant Peptide of the Human Melanoma Antigen Melan-A/MART-1 To A Peptide-Specific HLA-A*0201+ Cytotoxic Cell Line,” J Immunol, 157: 787-797 (1996); Rivoltini, L., et al., “Binding and Presentation of Peptides Derived From Melanoma Antigens MART-1 and Glycoprotein-100 by HLA-A2 Subtypes: Implications for Peptide-Based Immunotherapy,” J Immunol 156:3882-3891 (1996); Kawashima, I., “The Multi-Epitope Approach for Immunotherapy for Cancer: Identification of Several CTL Epitopes from Various Tumor-Associated Antigens Expressed on Solid Epithelial Tumors,” Hum Immunol 59:1-14 (1998)) settings.

### Example 6

### Peptide Composition for Prophylactic Uses

Vaccine compositions of the present invention are used to prevent infection or treat cancer in persons. For example, a polyepitopic peptide epitope composition containing multiple CTL and HTL epitopes is administered to individuals at risk for HCV infection. The composition is provided as a single lipidated polypeptide that encompasses multiple epitopes. The vaccine is administered in an aqueous carrier comprised of Freunds Incomplete Adjuvant. The dose of peptide for the initial immunization is from about 1 to about 50,000 fig for a 70 kg patient administered in a human dose volume. The initial administration of vaccine is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious as a prophylaxis against HCV infection.

Alternatively, the polyepitopic peptide composition can be administered as a nucleic acid in accordance with methodologies known in the art and disclosed herein.

The above discussion is provided to illustrate the invention but not to limit its scope. Other variants of the invention will be readily apparent to one of ordinary skill in the art and are encompassed by the appended claims. All publications, patents, and patent applications cited herein are hereby incorporated by reference.

TABLE 3

| | | Phenotypic frequency^{a} | | | | |
|---|---|---|---|---|---|---|
| Super- | | | Cau- | | | |
| type | Allele | Blacks | casians | Orientals | Hispanics | Average |
| A2 | A*0201 | 22.3 | 45.6 | 18.1 | 37.1 | 30.8 |
| | A*6802 | 12.7 | 1.8 | 0.0 | 4.2 | 4.7 |
| | A*0206 | 0.0 | 0.4 | 9.3 | 6.3 | 4.0 |
| | A*0207 | 0.0 | 0.0 | 11.0 | 0.0 | 2.7 |
| | A*0205 | 5.2 | 1.8 | 0.3 | 3.0 | 2.5 |
| | A*0203 | 0.0 | 0.0 | 8.8 | 0.0 | 2.2 |
| | A*0202 | 6.4 | 0.0 | 0.5 | 1.3 | 2.0 |
| | A*6901 | 0.0 | 0.7 | 0.3 | 1.3 | 0.6 |
| | A*0211 | 0.0 | 0.0 | 0.0 | 1.3 | 0.3 |
| | A*0212 | 0.0 | 0.0 | 0.3 | 0.8 | 0.3 |
| | A*0213 | 0.0 | 0.0 | 0.0 | 0.4 | 0.1 |
| | A*0214 | 0.0 | 0.0 | 0.0 | 0.0 | 0.0 |
| | Total | 43.1 | 48.2 | 45.0 | 51.9 | 47.1 |

^{a}Phenotypic frequencies were calculated from unpublished data provided by M. Fernandez-Vina and D. Mann. Frequencies greater than 5% are indicated by bold foot.

TABLE 4

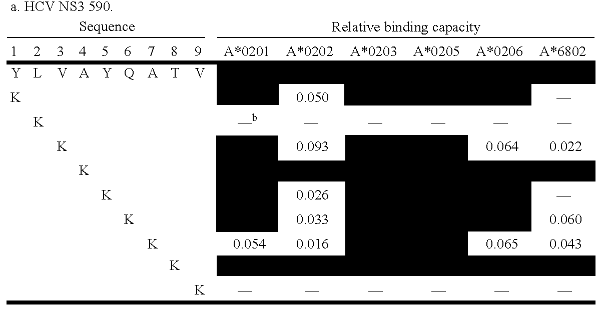

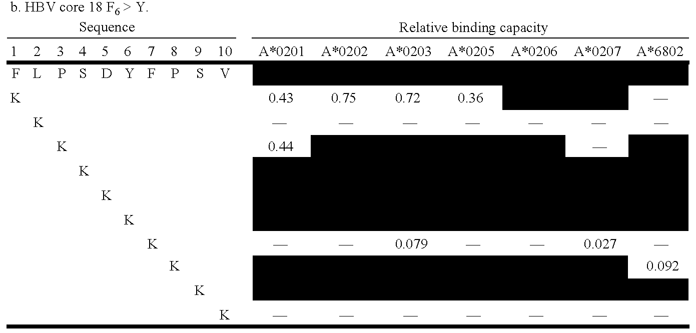

a) Binding capacities are expressed as ratios relative to the parent peptide. Peptides whose binding capacities are within 10-fold of the best binder are highlighted by shading, and are considered preferred; those whose relative binding capacities are 10-100-fold less than the best binder are considered tolerated.
b) A dash (“—”) indicates relative binding <0.01.

TABLE 5

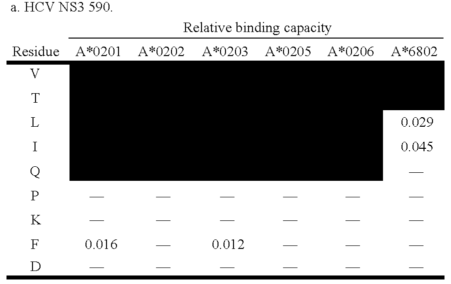

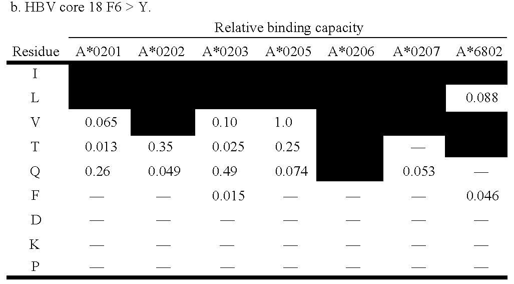

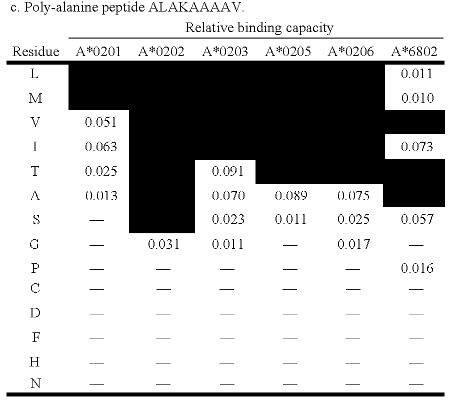

d Summary.

| | Allele/Peptide combinations^{b} | | | | % tolerated |
|---|---|---|---|---|---|
| Residue | Tested | Preferred | Tolerated | % preferred | or preferred |
| V | 19 | 17 | 2 | 89.5 | 100.0 |
| L | 19 | 16 | 3 | 84.2 | 100.0 |
| I | 19 | 16 | 3 | 84.2 | 100.0 |
| M | 6 | S | 1 | 83.3 | 100.0 |
| T | 19 | 14 | 4 | 73.7 | 94.7 |
| A | 6 | 2 | 4 | 33.3 | 100.0 |
| Q | 13 | 8 | 3 | 61.5 | 84.6 |
| S | 6 | 1 | 4 | 16.7 | 83.3 |
| G | 6 | 0 | 3 | 0.0 | 50.0 |
| F | 19 | 0 | 4 | 0.0 | 21.1 |
| P | 19 | 0 | 1 | 0.0 | 5.3 |
| C | 6 | 0 | 0 | 0.0 | 0.0 |
| K | 19 | 0 | 0 | 0.0 | 0.0 |
| N | 6 | 0 | 0 | 0.0 | 0.0 |
| D | 19 | 0 | 0 | 0.0 | 0.0 |

a) Binding Capacities are expressed as ratios relative to the related analog with the highest binding affinity for each individual molecule. Peptides whose relative binding capacities axe in the 1-0.1 range are highlighted by shading, and are considered preferred; those whose relative binding capadties are in the 0.1-0.01 range are considered tolerated. A dash (“—”) indicates relative binding <0.01.
b) Indicates the number of instances in which a given residue was associated with relative binding in the 1-0.1 range (preferred) or 0.1-0.01 range (tolerated).

TABLE 6

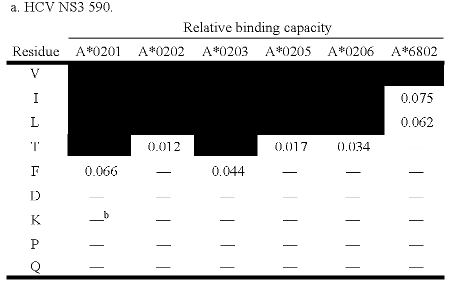

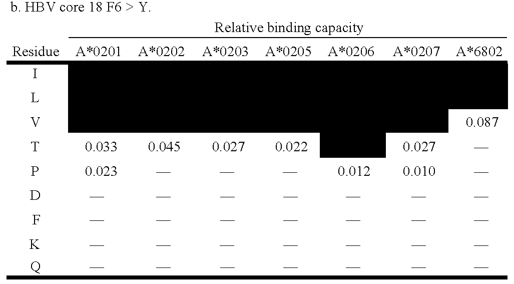

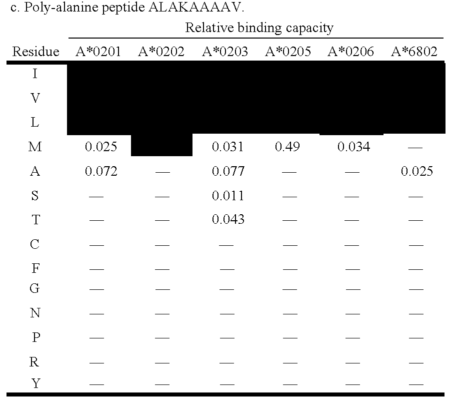

d Summary.

| | Allele/Peptide combinations^{b} | | | | % tolerated |
|---|---|---|---|---|---|
| Residue | Tested | Preferred | Tolerated | % preferred | or preferred |
| V | 19 | 19 | 0 | 100.0 | 100.0 |
| 1 | 19 | 18 | 1 | 93.3 | 100.0 |
| L | 19 | 14 | 5 | 66.7 | 100.0 |
| M | 6 | 1 | 4 | 20.0 | 83.3 |
| T | 19 | 3 | 9 | 20.0 | 63.2 |
| A | 6 | 0 | 3 | 0.0 | 50.0 |
| S | 6 | 0 | 1 | 0.0 | 16.7 |
| P | 19 | 0 | 3 | 0.0 | 15.8 |
| F | 19 | 0 | 2 | 0.0 | 10.5 |
| C | 6 | 0 | 0 | 0.0 | 0.0 |
| G | 6 | 0 | 0 | 0.0 | 0.0 |
| N | 6 | 0 | 0 | 0.0 | 0.0 |
| R | 6 | 0 | 0 | 0.0 | 0.0 |
| K | 13 | 0 | 0 | 0.0 | 0.0 |
| Y | 6 | 0 | 0 | 0.0 | 0.0 |
| D | 13 | 0 | 0 | 0.0 | 0.0 |
| Q | 13 | 0 | 0 | 0.0 | 0.0 |

a) Binding capacities are expressed as ratios relative to the related analog with the highest binding affinity for each individual molecule. Peptides whose relative binding capacities are in the 1-0.1 range are highlighted by shading, and are considered preferred; those whose reladve binding capacities are in the 0.1-0.01 range are considered tolerated. A dash (“—”) indicates relative binding <0.01.
b) Indicates the number of instances in which a given residue was associated with relative binding in the 1-0.1 range (preferred) or 0.1-0.01 range (tolerated).

TABLE 7
a. Binding as a function of peptide size.

| Peptide | | % Binding | |
|---|---|---|---|
| length | (n) | peptides | ARB^{a} |
| 8 | 171 | 3.5 | 0.072 |
| 9 | 2066 | 27.6 | 1.0 |
| 10 | 1451 | 17.8 | 0.27 |
| 11 | 179 | 14.5 | 0.20 |
| Total | 3867 | 22.2 | |

b. Binding as a function of main anchor motifs.

| | |
|---|---|
| Motif | % Binding |

| Position 2 | C-terminus | (n) | peptides | ARB |
|---|---|---|---|---|
| Preferred | Preferred | 526 | 48.7 | 1.0 |
| Preferred | Tolerated | 1446 | 28.4 | 0.31 |
| Tolerated | Preferred | 558 | 17.6 | 0.098 |
| non-tolerated | Preferred | 27 | 0.0 | 0.031 |
| Preferred | non-tolerated | 66 | 6.1 | 0.026 |
| Tolerated | Tolerated | 1337 | 7.1 | 0.026 |
| non-tolerated | Tolerated | 46 | 0.0 | 0.015 |
| non-tolerated | non-tolerated | 71 | 0.0 | 0.014 |
| Tolerated | non-tolerated | 105 | 0.0 | 0.013 |
| Total | | 4182 | 20.7 | |

^{a}ARB values are standardized to the peptide set carrying preferred residues in both primary anchor positions.

TABLE 8

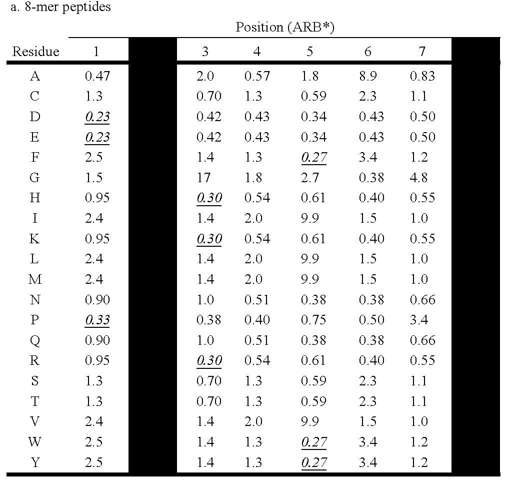

a) A panel of 9-mer peptides based on naturally occuring sequences from various viral, bacterial, or pathogen
origin was analyzed. All peptides had at least 1 preferred and 1 tolerated residues at the main anchor positions.
ARB values thrown were calculated as described in the materials and methods and are based on the grouping of
chemically similar (see e.g. ref. 6). At secondary anchor positions values corresponding to a 3-fold or greater
increase in binding capacity are indicated by increased font. Positive effects are further identified by boldes font,
and negative effects by underlined and italicized font. Main anchor positions are shaded, and residues determined
to be preferred or tolerated anchors are indicated by bold font. ARB values at the anchor positions were derived
from the analyses described in FIG. 1. To allow some of the values shown in the table as coefficients for
predictive algorithm, the values for non-tolerated anchor residues have been set to 0.001, equivalent to a
1000-fold reduction in bonding capacity to filter out non-motif peptides. The average geometric binding capacity
of the panel was 14420 aM.

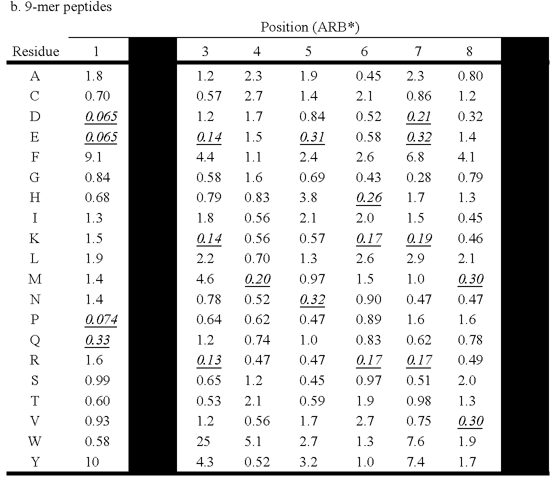

a) A panel of 1389 9-mer peptides based on naturally occuring sequences from variuous viral, bacterial, or
pathogen origin was analyzed. All peptides had at least 1 preferred and I tolerated residues at the main anchor
positives. ARV values shown were calculated as described in the materials and methods, and were derived for
each residue considered individually. At secondary anchor positions values corresponding to a 3-fold or greater
increase in binding capacity are indicated by increased font. Positive effects are further identified by bolded
font, and negative effects by underlined and italicized font. Main anchor positions are shaded, and residues
determined to be preferred or tolerated anchors are indicated by bold font. ARB values at the anchor positions
were derived from the analyses described in FIG. 1. To allow use of the values shown in this table as coefficients
for predicitve alogorithm, the values for non-tolerated anchor residues have been set to 0.001, equivalent to a
1000-fold reduction in binding capacity, to filter out non-motif peptides. The average geometric binding capacity
of the panel was 1581 aM.

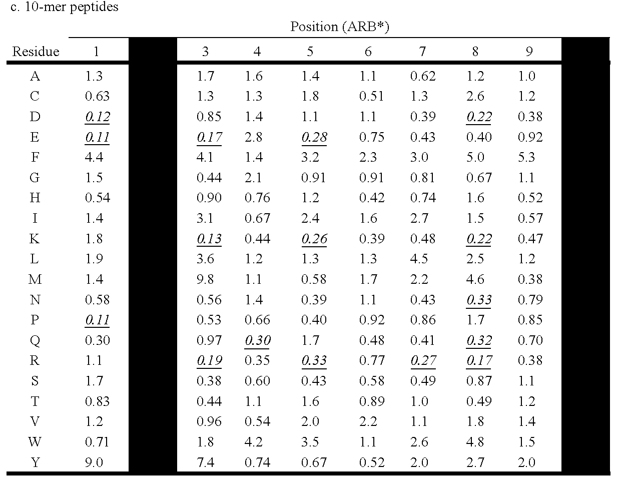

a) A panel of 953 10-mer peptides based on naturally occuring sequences from various viral, bacterial,
or pathogen origin was analyzed. All peptides had at least 1 preferred and 1 tolerated residue at the main
anchor positions. ARB values shown were calculated as described in the materials and methods, and were
derived for each residue considered individually. At secondary anchor positions values corresponding to a
3-fold or greater increase in binding capacity are indicated by increased font. Positive effects are
further identified by bolded font, and negative effects by underlined and itialicized font. Main anchor
positions are shaded, and residues determined to be preferred or tolerated anchors are indicated by bold
font. ARB values at the anchor positions were derived from the analyses described by FIG. 1. To allow use of
the values shown in the table are coefficients for predictive algorithm, the
values for non-tolerated anchor residues have been set to 0.001, equivalent to 1000-fold residues in
binding capacity, to filter ou non-motif peptides. The average geometric binding capacity of the panel was
3155 aM.

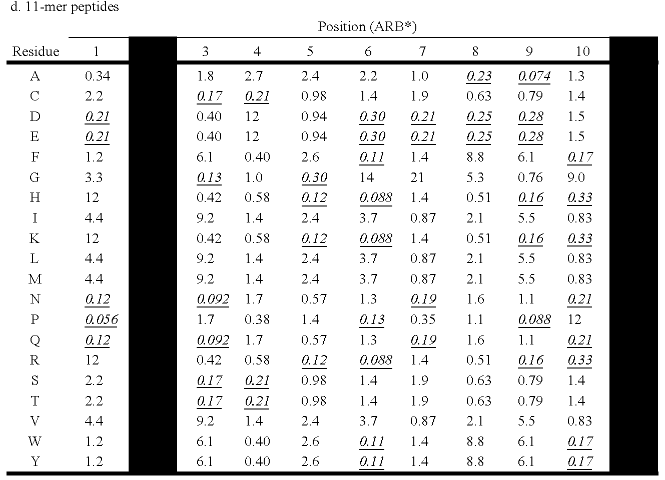

a) A panel of 95 11-mer peptides based on naturally occuring sequences from various viral, bacterial, or pathogen
origin was analyzed. All peptides had at least 1 preferred and 1 tolerated residue at the main anchor positions.
ARB values shown were calculated as described in the materials and methods, and are based on the grouping of
chemically similar residues (see e.g. ref 6). At secondary anchor positions values corresponding to a 3-fold or
greater increase in binding capacity are indicated by increased font. Positive effects are further identified by
bolded font, and negative effects by underlined and italicized font. Main anchor positions are shaded, and residues
determined to be preferred or tolerated anchors are indicated by bold font. ARB values at the anchor positions
were derived from the analyses described in FIG. 1. To allow use of the values shown in this table as coefficients
for predictive algorithm, the values for non-tolerated anchor residues have been set to 0.001, equivalent to a
1000-fold reduction in binding capacity, to filter out non-motif peptides. The average geometric binding capacity
of the panel was 3793 aM.

TABLE 9

| | | |
|---|---|---|
| Peptide length | (n) | ARB^{a} |

a. A*0202

| | | |
|---|---|---|
| 8 | 6 | 0.050 |
| 9 | 268 | 0.79 |
| 10 | 120 | 1.0 |
| 11 | 16 | 0.90 |
| Total | 410 | |

b. A*0203

| | | |
|---|---|---|
| 8 | 6 | 0.11 |
| 9 | 272 | 1.0 |
| 10 | 122 | 0.75 |
| 11 | 16 | 0.36 |
| Total | 416 | |

c. A*0206

| | | |
|---|---|---|
| 8 | 6 | 0.066 |
| 9 | 268 | 1.0 |
| 10 | 120 | 0.38 |
| 11 | 16 | 0.66 |
| Total | 410 | |

d. A*6802

| | | |
|---|---|---|
| 8 | 6 | 0.071 |
| 9 | 268 | 1.0 |
| 10 | 120 | 0.60 |
| 11 | 16 | 0.47 |
| Total | 410 | |

^{a}ARB values are standardized to the peptide set carrying preferred residues in both primary anchor positions.

TABLE 10

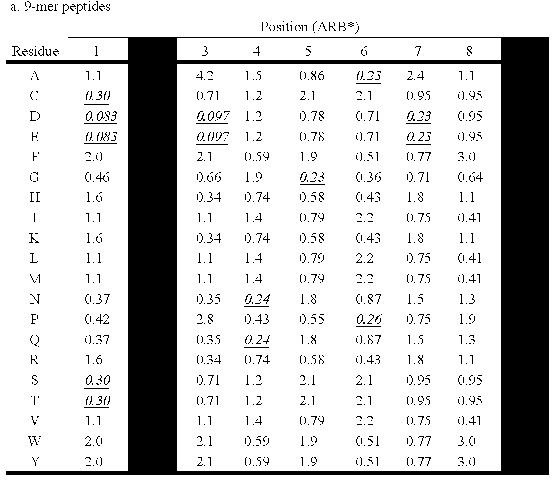

a) A panel of 268 9-mer peptides based on naturally occuring sequences from various viral, bacterial, or
pathogen origin waes analyzed. All peptides had at least 1 preferred and 1 tolerated residue at the main
anchor positions. ARB values shown were calculated as described in the materials and methods, and
are based on the grouping of chemically similar residues (see e.g. ref. 6). At secondary anchor
positions values corresponding to a 3-fold or greater increase in bonding capacity are indicated by
increased font. Positive effects are further identified by bolded font, and negative effects by underlined
and italicized font. Main anchor positions are shaded, and residues determined to be preferred or
tolerated anchors are indicated by bold font. ARB values at the anchor positions were derived from the
analyses described in FIGS. 3 and 4. To allow use of the values shown in the table as coefficients for
predictive alorigthm the values for non-tolerated anchor residues have been set to 0.001, equivalent to
a 1000-fold reduction in binding capacity, to filter out most motif peptides. The average geometric binding
capacity to the panel was 401 aM.

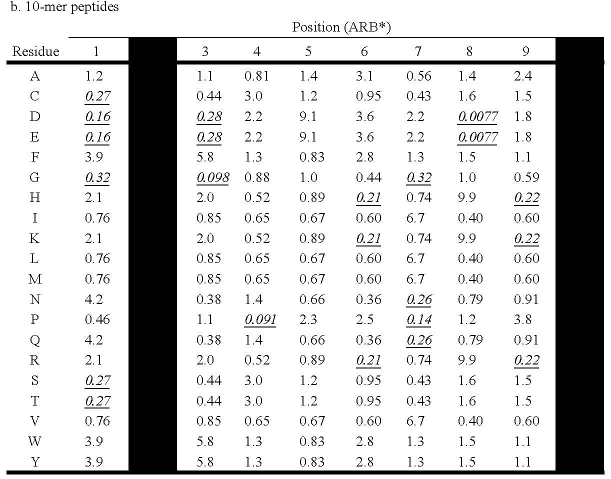

a) A panel of 120 10-mer peptides based on naturally occuring sequences from various viral, bacterial, or
pathogen origin was analyzed. All peptides had at least 1 preferred and 1 tolerated residue at the main
anchor positions. ARB values shown were calculated as described in the materials and methods, and are
based on the grouping of chemically similar residues (see e.g. ref. 6). At secondary anchor positions
values corresponding to a 3-fold or greater increase in binding capacity are indicated by increased font.
Positive effects are further identified by bolded font, and negative effects by underlined and italicized
font. Main anchor positions are shaded, and residues determined to be preferred or tolerated anchors are
indicated by bold font. ARB values at the anchor positions were derived from the analyses described in
FIGS. 3 and 4. To allow use of the values shown in this table as coefficients for predictive algorithm,
the values for non-tolerated anchor residues have been set to 0.001, equivalent to a 1000-fold reduction
in binding capacity, to filter out non-motif peptides. The average geometric binding capacity of the
panel was 342 aM.

TABLE 11

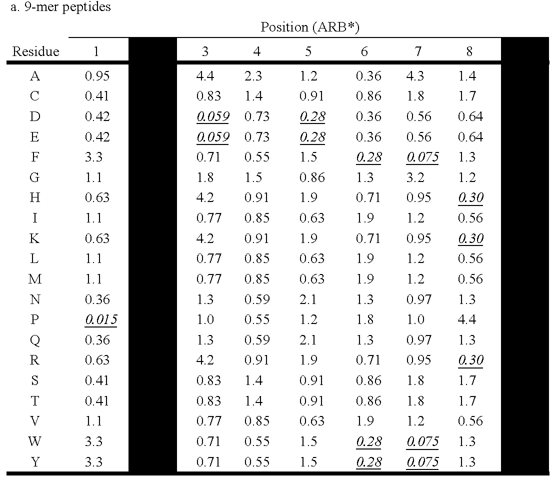

a) A panel of 272 9-mer peptides based on naturally occuring sequences from various viral,
bacterial or pathogen origin was analyzed. All peptides had at least 1 preferred and 1 tolerated
residue at the mass anchor positions. ARB values shown were calculated as described in the
materials and methods and are based on the grouping of chemically similar residues (see e.g. ref 6).
At secondary anchor positions values corresponding to a 3-fold or greater increase in binding
capacity are indicated by increased font. Positive effects are further indentified by bolded font,
and negative effects by underlined and italicized font. Main anchor positions are shaded, and
residues determined to be preferred or tolerated anchors are indicated by bold font. ARB values
at the anchor positions were derived from the analyses described in FIGS. 3 and 4. To allow use
of the values shown in this table as coefficients for predictive algorithms, the values for
non-tolerated anchor residues have been set to a 1000-fold reduction in binding capapcity to filter
out non-motif peptides. The average geometric binding capacity of the panel was 85 nM.

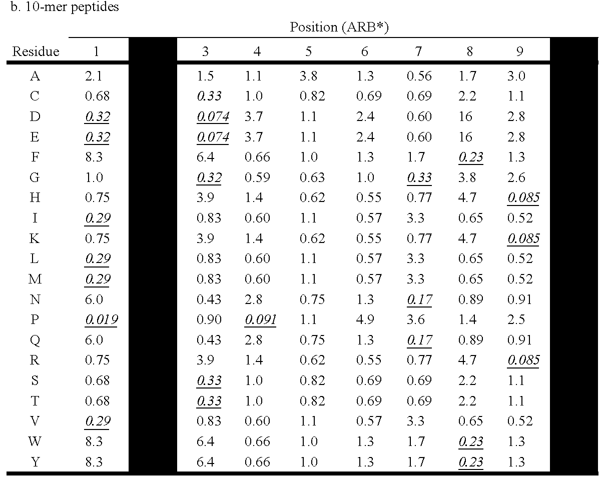

a) A panel of 122 10-mer peptides based on naturally occuring sequences from various viral,
bacterial or pathogen origin was analyzed. All peptides had at least 1 preferred and 1 tolerated
residue at the main anchor positions. ARB values shown were calculated as described in the
materials and methods, and are based on the grouping of chemically similar residues (see e.g.
ref 6). At secondary anchor positions values corresponding to a 3-fold or greater increase in
binding capacity are indicated by increased font. Positive effects are further indentified by
bolded font, and negative effects by underlined and italicized font. Main anchor positions are
shaded, and residues determined to be preferred or tolerated anchors are indicated by bold font.
ARB values at the anchor positions were derived from the analyses described in FIGS. 3 and 4.
To allow use of the values shown in this table as coefficients for predictive algorithms, the
values for non-tolerated anchor residues have been set to a 0.001, equivalent to a 1000-fold
reduction in binding capapcity to filter out non-motif peptides. The average geometric binding
capacity of the panel was 89 nM.

TABLE 12

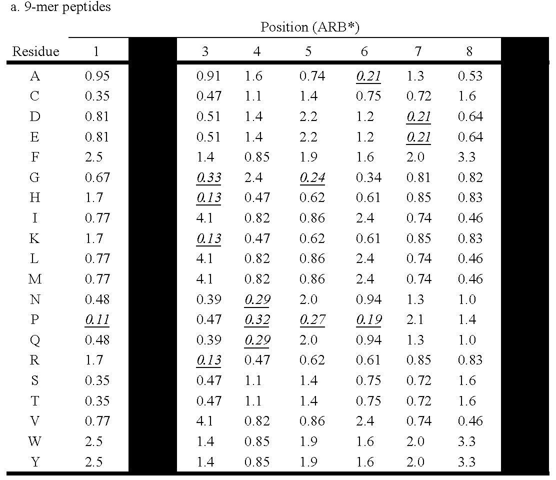

a) A panel of 264 9-mer peptides based on naturally occuring sequences from various viral,
bacterial, or pathogen origin was analyzed. All peptides had at least 1 preferred and 1 tolerated
residue at the main anchor positions. ARB values shown were calculated as described in the
materials and methods and are based on the grouping of chemically similar residues (see e.g. ref 6).
At secondary anchor positions values corresponding to a 3-fold or greater increase in binding
capacity are indicated by increased font. Positive effects are further indentified by bolded font,
and negative effects by underlined and italicized font. Main anchor positions are shaded, and
residues determined to be preferred or tolerated anchors are indicated by bold font. ARB values
at the anchor positions were derived from the analyses described in FIGS. 3 and 4. To allow use
of the values shown in this table as coefficients for predictive algorithms, the values for
non-tolerated anchor residues have been set to a 0.001 equivalent to a 1000-fold reduction in
binding capapcity to filter out non-motif peptides. The average geometric binding capacity of
the panel was 85 nM.

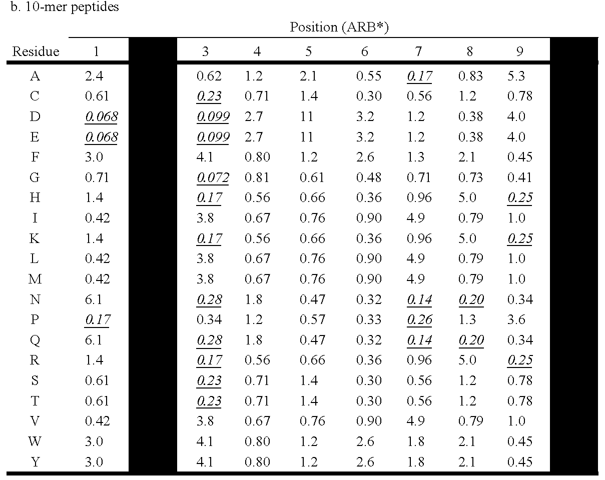

a) A panel of 120 10-mer peptides based on naturally occuring sequences from various viral,
bacterial, or pathogen origin was analyzed. All peptides had at least 1 preferred and 1 tolerated
residue at the main anchor positions. ARB values shown were calculated as described in the
materials and methods and are based on the grouping of chemically similar residues (see e.g. ref 6).
At secondary anchor positions values corresponding to a 3-fold or greater increase in binding
capacity are indicated by increased font. Positive effects are further indentified by bolded font,
and negative effects by underlined and italicized font. Main anchor positions are shaded, and
residues determined to be preferred or tolerated anchors are indicated by bold font. ARB values
at the anchor positions were derived from the analyses described in FIGS. 3 and 4. To allow use
of the values shown in this table as coefficients for predictive algorithms, the values for
non-tolerated anchor residues have been set to a 0.001 equivalent to a 1000-fold reduction in
binding capapcity to filter out non-motif peptides. The average geometric binding capacity of
the panel was 643 nM.

TABLE 13

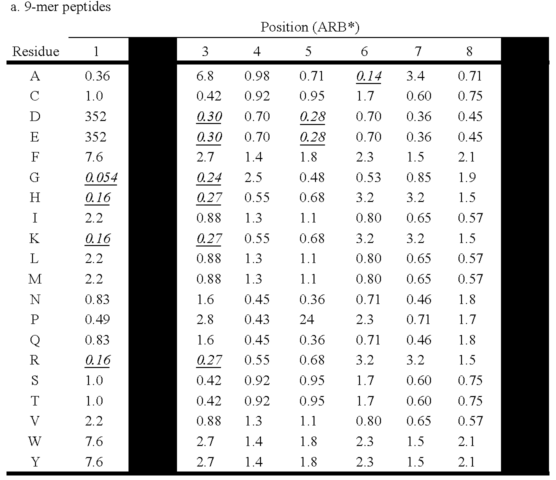

a) A panel of 268 9-mer peptides based on naturally occuring sequences from various viral,
bacterial, or pathogen origin was analyzed. All peptides had at least 1 preferred and 1 tolerated
residue at the main anchor positions. ARB values shown were calculated as described in the
materials and methods and are based on the grouping of chemically similar residues (see e.g. ref 6).
At secondary anchor positions values corresponding to a 3-fold or greater increase in binding
capacity are indicated by increased font. Positive effects are further indentified by bolded font,
and negative effects by underlined and italicized font. Main anchor positions are shaded, and
residues determined to be preferred or tolerated anchors are indicated by bold font. ARB values
at the anchor positions were derived from the analyses described in FIGS. 3 and 4. To allow use
of the values shown in this table as coefficients for predictive algorithms, the values for
non-tolerated anchor residues have been set to a 0.001 equivalent to a 1000-fold reduction in
binding capapcity to filter out non-motif peptides. The average geometric binding capacity of
the panel was 838 nM.

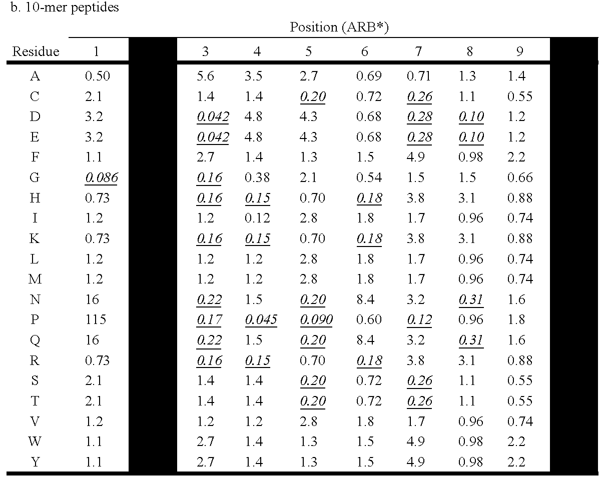

a) A panel of 120 10-mer peptides based on naturally occuring sequences from various viral,
bacterial, or pathogen origin was analyzed. All peptides had at least 1 preferred and 1 tolerated
residue at the main anchor positions. ARB values shown were calculated as described in the
materials and methods and are based on the grouping of chemically similar residues (see e.g. ref 6).
At secondary anchor positions values corresponding to a 3-fold or greater increase in binding
capacity are indicated by increased font. Positive effects are further indentified by bolded font,
and negative effects by underlined and italicized font. Main anchor positions are shaded, and
residues determined to be preferred or tolerated anchors are indicated by bold font. ARB values
at the anchor positions were derived from the analyses described in FIGS. 3 and 4. To allow use
of the values shown in this table as coefficients for predictive algorithms, the values for
non-tolerated anchor residues have been set to a 0.001 equivalent to a 1000-fold reduction in
binding capapcity to filter out non-motif peptides. The average geometric binding capacity of
the panel was 1055 nM.

TABLE 14

| 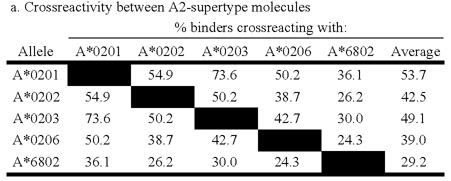 | | | | | | | |
|---|---|---|---|---|---|---|---|
| b. Degeneracy of A*0201 binders. | | | | | | | |
| | A2-supertypes alleles bound (% of peptides) | | | | | | |
| A*201 affinity | 0 | 1 | 2 | 3 | 4 | 5 | >=3 |
| <=20 | 0.0 | 0.0 | 3.5 | 17.5 | 36.8 | 42.1 | 96.5 |
| <=100 | 0.0 | 3.6 | 11.2 | 21.4 | 34.7 | 29.1 | 85.2 |
| <=500 | 0.0 | 7.1 | 20.1 | 25.1 | 28.3 | 19.3 | 72.8 |
| >500 | 40.0 | 33.3 | 16.7 | 10.0 | 0.0 | 0.0 | 10.0 |

What is claimed is:

1. A method for identifying a HLA-A2 supermotif-restricted peptide, comprising:
contacting a peptide consisting of 8-11 amino acids, wherein the amino acid at position two from the N-terminus of the peptide is L, I, V, M, A, T, or Q and the C-terminal amino acid is L, I, V, M, A, or T, with three or more of the HLA molecules encoded by A*0201, A*0202, A*0203, A*0204, A*0205, A*0206, A*0207, A*6802, and A*6901 alleles;
measuring IC₅₀ values; and
identifying a peptide that binds at least three HLA molecules with an IC₅₀ value less than 500 nM as a HLA-A2 supermotif restricted peptide.

2. The method of claim 1, wherein the amino acid at position two of the peptide is V, A, T, or Q.

3. The method of claim 1, wherein the amino acid at position two of the peptide is L, A, M, or Q.

4. The method of claim 1, wherein the amino acid at position two of the peptide is I or Q.

5. The method of claim 57, wherein the C-terminal amino acid is L, I, V, M, A, or T.

6. The method of claim 1, wherein the C-terminal amino acid is T.

7. The method of claim 1, wherein the peptide is derived from an HIV antigen, HBV antigen, HCV antigen, HPV antigen, PSA antigen, Epstein-Barr virus antigen, KSHV antigen, Lassa virus antigen, MT antigen, p53 antigen, CEA antigen, TSA antigen, MAGE antigen, or Her2/neu antigen.

8. A method for identifying an immunogenic HLA-A2 supermotif-restricted peptide, comprising:
contacting a peptide consisting of 8-11 amino acids, wherein the amino acid at position two from the N-terminus of the peptide is L, I, V, M, A, T, or Q and the C-terminal amino acid is L, I, V, M, A, or T to form peptide/HLA-A2 complexes, with three or more of the HLA molecules encoded by A*0201, A*0202, A*0203, A*0204, A*0205, A*0206, A*0207, A*6802, and A*6901 alleles;
determining whether the peptide/HLA-A2 complexes induce a CTL response, and
identifying a peptide that induces a CTL response in complex with at least three of the HLAs as a HLA-A2 supermotif restricted peptide.

9. The method of claim 8, wherein the amino acid at position two of the peptide is V, A, T, or Q.

10. The method of claim 8, wherein the amino acid at position two of the peptide is L, I, M, or Q.

11. The method of claim 8, wherein the amino acid at position two of the peptide is I or Q.

12. The method of claim 8, wherein the C-terminal amino acid is L, I, V, M, A, or T.

13. The method of claim 8, wherein the C-terminal amino acid is T.

14. The method of claim 8, wherein the peptide is derived from an HIV antigen, HBV antigen, HCV antigen, HPV antigen, PSA antigen, Epstein-Barr virus antigen, KSHV antigen, Lassa virus antigen, MT antigen, p53 antigen, CEA antigen, TSA antigen, MAGE antigen, or Her2/neu antigen.

15. A method for making a HLA-A2 supermotif-restricted peptide, comprising:
providing an amino acid sequence of an antigen of interest;
identifying within the sequence a putative T-cell epitope, wherein the putative epitope consists of 8-11 amino acids, wherein the amino acid at position two from the N-terminus of the epitope is L, I, V, M, A, T, or Q and the C-terminal amino acid is L, I, V, M, A, or T,
preparing one or more peptide fragments of the antigen of interest that comprise the epitope;
contacting the peptide with three or more of the HLA molecules encoded by A*0201, A*0202, A*0203, A*0204, A*0205, A*0206, A*0207, A*6802, and A*6901 alleles;
measuring IC₅₀ values; and
selecting a peptide that binds at least three HLA molecules with an IC₅₀ value less than 500 nM as a HLA-A2 supermotif-restricted peptide.

16. The method of claim 15, wherein the amino acid at position two of the peptide is V, A, T, or Q.

17. The method of claim 15, wherein the amino acid at position two of the peptide is L, I, M, or Q.

18. The method of claim 15, wherein the amino acid at position two of the peptide is I or Q.

19. The method of claim 15, wherein the C-terminal amino acid is L, I, V, M, A, or T.

20. The method of claim 15, wherein the C-terminal amino acid is T.

21. The method of claim 15, wherein the antigen is HIV, HBV, HCV, HPV, PSA, Epstein-Barr virus, KSHV, Lassa virus, MT, p53, CEA, TSA, MAGE, or Her2/neu.

22. A method for making an immunogenic HLA-A2 supermotif-restricted peptide, comprising:
providing an amino acid sequence of an antigen of interest;
identifying within the sequence a putative T-cell epitope, wherein the putative epitope consists of 8-11 amino acids, wherein the amino acid at position two from the N-terminus of the epitope is L, I, V, M, A, T, or Q and the C-terminal amino acid is L, I, V, M, A, or T,
preparing one or more peptide fragments of the antigen of interest that comprise the epitope;
determining whether the peptide/HLA-A2 complexes induce a CTL response, and
selecting a peptide that induces a CTL response in complex with at least three of the HLAs as a HLA-A2 supermotif restricted peptide.

23. The method of claim 22, wherein the amino acid at position two of the peptide is V, A, T, or Q.

24. The method of claim 22, wherein the amino acid at position two of the peptide is L, I, M, or Q.

25. The method of claim 22, wherein the amino acid at position two of the peptide is I or Q.

26. The method of claim 22, wherein the C-terminal amino acid is L, I, V, M, A, or T.

27. The method of claim 22, wherein the C-terminal amino acid is T.

28. The method of claim 22, wherein the antigen is HIV, HBV, HCV, HPV, PSA, Epstein-Barr virus, KSHV, Lassa virus, MT, p53, CEA, TSA, MAGE, or Her2/neu.
